(19) United States
(12) Patent Application Publication
(10) Pub. No.: US 2010/0113569 A1
(43) Pub. Date: May 6, 2010

(54) ISOLATED DNA FRAGMENT OF THE HUMAN A33 PROMOTER AND ITS USE TO CONTROL THE EXPRESSION OF A HETEROLOGOUS GENE IN TUMOR CELLS

(75) Inventors: Osvaldo Luis Podhajcer, Buenos Aires (AR); Eduardo Gustavo Alfredo Cafferata, Buenos Aires (AR); Maria Veronica Lopez, La Plata (AR); Diego Luis Viale, Buenos Aires (AR); Daniela Rita Maccio, Cordoba (AR)

Correspondence Address: FISH & RICHARDSON PC, P.O. BOX 1022, MINNEAPOLIS, MN, 55440-1022, US

(73) Assignee: INIS BIOTECH LLC, Kent County, DE (US)

(21) Appl. No.: 12/298,691

(22) PCT Filed: Apr. 26, 2007
(86) PCT No.: PCT/US07/67567

§ 371 Date: Jan. 11, 2010

(51) Int. Cl. A61K 31/7088; C07H 21/04; C12N 15/63; C12N 5/10

(52) U.S. Cl. 514/44 R; 536/23.1; 536/23.2; 536/23.5; 435/320.1; 435/325

(57) ABSTRACT

An isolated DNA sequence that corresponds to a region of the human A33 gene promoter from base pair −105 to base pair +307, able to control the expression of a heterologous gene of interest, that may be used in conjunction with any other regulatory sequence, including sequences responsive to stress such as radiation, hypoxia and free radical formation. Constructs and viral vectors for recombinant DNA expression are provided, which include this sequence of human A33 gene promoter and, operably linked thereto, at least one heterologous gene. The regulatory sequence(s) control(s) the expression of at least one heterologous gene in cells including colorectal cancer cells. Pharmaceutical compositions and methods for treating colorectal cancer are also described.

### TECHNICAL FIELD

This invention relates to genetic and cancer therapies, and more particularly to isolated DMA sequences having the ability to control the expression of a gene of interest in, for example, a tumor cell. More specifically, the present invention is concerned with vectors that contain an A33 promoter associated with a gene of interest and with pharmaceutical compositions and their use in colorectal cancer therapy.

This invention relates to cancer therapies, and more particularly to an oncolytic adenovirus useful in the treatment of colon cancer.

### BACKGROUND OF THE INVENTION

Colorectal cancer is one of the leading causes of death by malignant tumors, and approximately 50% of patients with colorectal cancer develop recurrence within, the five years following treatment of the primary cancer. In the majority of these cases, the cancer becomes a chronic illness with a life expectancy of approximately five years. In the more advanced stages of the illness, there can be metastases to the lungs, liver, ovary and hone (Taylor et al., Ann. Surg. Oncol., 9:177-185, 2002).

The first symptom of the illness can be the appearance of blood in the feces, by which point the cancer is already present in a very advanced stage. Patients do not usually respond to radiation therapy, and less titan 20% respond partially to chemotherapy drugs such as fluoracil 5. The lack of response to conventional treatments such as chemotherapy, radiotherapy or immunotherapy has contributed to the interest in finding a cure for this cancer, genetic therapy being one relevant strategy for the treatment of cancer in general. While there is a great deal of preclinical work and many clinical trials related to cancer, only a relatively few of these trials feature genetic therapies for the treatment of colorectal cancer or hepatic metastasis due to colorectal cancer (Friedmann, Acta Paediair. 85:1261-1265, 1996; Sangro et al., J. Clin. Oncol. 22:1389-3397, 2004; Sung et al., Mol. Ther. 4:182-191, 2001; and Habib et al., Human Gene Ther. 12:21.9-226, 2001).

### SUMMARY

The present invention is based, in part, on our work with the A33 antigen, which is selectively upregulated in certain malignant cells, including colorectal and gastric cancer cells. Accordingly, the invention features DNA sequences and, more generally, nucleotide sequences, that can be incorporated into expression vectors and used in the diagnosis and treatment of colorectal and gastric cancers. The sequences include those in which a colon cancer cell-specific regulatory sequence (e.g., a portion of an A33 antigen gene sequence) is used to drive the expression of a heterologous protein (i.e., a protein other than an A33 antigen). The heterologous protein can be one useful as a label or diagnostic marker or one useful as a therapeutic agent. One object, therefore, of the present invention is to provide an isolated DNA sequence, with promoter activity, capable of controlling the expression of a gene of interest, especially in a turner cell and particularly in a colorectal or gastric cancer cell.

The present invention provides an isolated fragment of DNA that includes a portion of the human A33 regulatory sequence, as described further below, that is able to control the expression of a heterologous gene of interest in a tumor cell, especially a tumor cell in which the A33 antigen is expressed or overexpressed. More specifically, the present invention features an isolated DNA sequence that includes a first regulatory sequence including SEQ ID NO:1 or a biologically-active variant thereof. The regulatory sequence, or the biologically active variant thereof when operably linked to a heterologous gene of interest, is capable of driving the expression of the heterologous gene of interest. For ease of reading, we do not continue to repeat the term “or a biologically active variant thereof” at every opportunity. It is to be understood that where an A33 regulatory sequence (e.g., SEQ ID NO:1) can be used, a biologically active variant thereof can also be used. The biologically active variant of the first regulatory sequence can be, or can include, a sequence that is at least 50% identical to SEQ ID NO:1 (additional variants are described further below), with the differences being caused by. an insertion, deletion or substitution of one or more nucleotide.

The sequences can include more than one regulatory sequence, and we may refer to these as “first” and “second” sequences. Where a second sequence is present in addition to an A33 regulatory sequence (e.g., SEQ ID NO:1), that second sequence can be one that is activated by a stressful stimulus. These sequences include a hypoxia response element or a promoter naturally associated with a stress protein, an NFkB response element, and a reactive oxygen species response element (e.g., the −80 bp to −50 bp of the human VEGF promoter) and the CArG motifs present in the Egr-1 promoter (Greco et al., Cancer Gene Ther. 12:655-662, 2005).

Any of the sequences described herein (e.g., SEQ ID NO:1 or constructs in which SEQ ID NO:1 is operably linked to a heterologous gene sequence) can be incorporated into an expression vector, such as a plasmid cosmid, an artificial chromosome, or a viral vector such as an adenoviral vector, or an adenoviral-associated vector. These types of vectors are well known in the art and can incorporate and be used to express the specific sequences described herein. As described further below, the adenoviral vector can constitute an oncolytic adenovirus.

The invention also features isolated cells that include a DMA or nucleic acid sequence described herein or an expression vector described herein.

The regulatory sequences (e.g., a sequence including SEQ ID NO:1 or a biologically active variant thereof) can be operably linked to a heterologous gene of interest, and these nucleic acid sequences, expression vectors containing them, and cells containing them are also within the present invention. Just as the regulatory sequences can include biologically active variants of an A33 sequence, they can also (or can alternatively) include biologically active variants of the heterologous gene of interest. The heterologous gene of interest can encode a therapeutic gene product such as an adenoviral protein (e.g., E1A), a pro-apoptotic protein (e.g., Bak, Bax, SIVA, Par-4, a Bcl-2, thymidine kinase, or a caspase), a tumor necrosis factor, or an interleukin (e.g., IL-2, IL-10, IL-12, or IL-23). Where an adenoviral protein is encoded, it can be that of any adenoviral serotype. Similarly, where an adenoviral vector is made and used, it can be an adenovirus of any serotype (e.g., serotype 2, serotype 5, serotype 3, or serotype 35).

Any of the present sequences can also include an internal ribosome entry site (IRES). IRESs have come to be understood as nucleic acid sequences that allow for translation initiation at an internal site on an mRNA.

The sequences, expression vectors, and cells described herein can be included as the active ingredient (or an active ingredient) in a pharmaceutical composition. The compositions can be formulated in any manner known to be appropriate for administering cancer therapeutics. These include formulations for either oral or parenteral administration (e.g., oral administration, intravenous administration, or intraarterial administration (e.g., intrahepatic artery administration)). The pharmaceutical compositions can also be formulated for direct administration, to a cancerous site in the gastrointestinal tract.

Also within the invention is the use of an isolated DNA sequence, nucleic acid sequence, expression vector or cell, having the features described herein, in the preparation of a medicament. The medicament can be one useful for the treatment of cancer (e.g., an A33 antigen-positive cancer such as colorectal cancer or gastric cancer).

The methods of the invention include methods of making the present isolated DNA sequences or nucleic acid sequences, expression vectors, and cells as well as methods of treating a patient who has a cancer amenable to treatment with these agents. For example, the patient may be one who has colorectal cancer or who is considered at risk of developing colorectal cancer. The methods can be carried out by administering to the patient a nucleic acid sequence described herein (e.g., a sequence including a first regulatory sequence that is, or that includes, SEQ ID NO:1, or a biologically active variant thereof and, operably linked thereto, a heterologous gene encoding an anti-colorectal cancer agent). The patient can be a human and the method can further include the step of identifying the human in need of treatment. As noted, the heterologous gene can encode an E1A, and the nucleic acid sequence can be contained within an expression vector (e.g., an adenoviral vector). The methods of treatment can further include subjecting the patient to a second treatment regime, which may also be directed toward eradicating the cancer. For example, the second treatment regime can include administration of an adjuvant chemotherapeutic agent such as 5-fluorouracil (5-FU), capecitabine (Xeloda™), leucovorin (LV; folinic acid), or oxaliplatin (Eloxatin™).

While we refer to “treatment” or to “treating” a patient, and while treatment may result in a complete remission or cure of the cancer, other beneficial outcomes are considered to be successful treatments as well. For example, a patient is successfully treated if the present methods result in an increase in life expectancy, an increase in quality of life (by, for example, reducing the symptoms of the cancer), a reduced risk of cancer recurrence, a reduced risk of metastasis, and the like.

More specifically, the invention provides an isolated DNA sequence that comprises the polynucleotide sequence SEQ ID NO:1, which corresponds to a region of the human A33 gene promoter from base pair −105 to base pair +307, or a biologically active fragment or other variant of this polynucleotide sequence that has been modified by the insertion, substitution, or deletion of one or more nucleotides. The protein normally associated with (i.e., normally expressed under the direction of) the A33 promoter is a membrane glycoprotein. Where the A33 regulatory sequence described herein is not contiguous with the A33 coding sequence, the A33 regulatory sequence is “isolated.”

Another aspect of the invention is that it provides an isolated DMA construct of recombinant expression that comprises the promoter sequence of the invention operably linked to a heterologous gene of interest.

An additional aspect of the invention is that the polynucleotide sequence SEQ ID NO:1 corresponding to a region of the human A33 gene promoter can be further linked with any other promoter sequence, such as sequences for response to irradiation, hypoxia, free radicals, etc.

Another relevant aspect of the present invention is that it provides a recombinant expression viral vector, containing the previously defined promoter DMA of the invention, where the DNA sequence is found to be operably linked to a gene of interest.

The invention also provides a method of expressing foreign DNA in a host cell, involving the introduction into the host cell of a recombinant expression DNA construct of the intervention that comprises the DNA promoter molecule or a recombinant viral vector of the invention that includes the DNA promoter module of polynucleotide sequence SEQ ID NO:1, operationally linked to a foreign DNA that encodes a desired polypeptide or RNA, in which this DNA is expressed.

The invention also provides a method for treating a colorectal tumor, in a patient suffering from this disease, which consists of administering to the patient an effective quantity of a pharmaceutical composition comprising a recombinant expression DNA construct or a recombinant expression viral vector, which includes the promoter sequence of the invention and is able to control the expression of a therapeutic gene and/or the viral replication operably linked to this.

As previously defined, an aspect of the present invention is that it provides an isolated DNA sequence that includes the polynucleotide sequence SEQ ID NO:1, which corresponds to a region of the human A33 gene promoter, from base pair −105 to base pair +307, or a fragment or variant of this polynucleotide sequence that has been modified by the insertion, substitution or deletion of one or more nucleotides, that has a substantially equivalent function.

The term “isolated,” as used herein, signifies substantially separated or purified with respect to the contaminating sequences in the cell or organism, in which, the nucleic acid is present naturally, and includes nucleic acids purified by standard purification techniques as well as nucleic acids prepared by recombinant technology or chemical synthesis.

The term “variant,” as used herein, refers to a DNA molecule in which the sequence of nucleotides is substantially identical to the sequence established as SEQ ID NO:1. The variant may be achieved by modifications such as insertion, substitution or deletion of one or more nucleic acids, if such modifications are neutral mutations and do not affect the functioning of the DNA molecule or if the DNA molecule functions as described further below.

A “fragment” of a nucleic acid sequence, as used herein, is a portion of nucleic acids that is less than the complete length and includes at least a minimum length capable of being hybridized specifically with the nucleic acid sequence of the present invention under stringent conditions, this fragment maintaining the biological properties required in the present invention.

A “heterologous gene”, as used here, indicates a DNA sequence that codes an amino acid sequence or a protein of interest linked to another DNA sequence, where this association is not found naturally.

The term “therapeutic gene”, as used herein, Indicates a DNA sequence that codes a sequence of ammo acids or a protein, capable of exercising a direct or indirect therapeutic effect on the host cells. For the present invention, the preferred host cells are colon tumor cells.

The following specific embodiments are within the scope of the present invention:

An isolated DNA sequence mat includes the polynucleotide sequence SEQ ID NO:1, which corresponds to a region of the human gene A33 promoter from base pair −105 to base pair +30 or a fragment or variant of this polynucleotide sequence that has been modified by the insertion, substitution or deletion of one or more nucleotides, which has a substantially equivalent function;

A sequence with promoter activity, in accordance with the isolated DNA sequence described immediately above, capable of controlling the expression of a heterologous gene of interest, operably linked to it;

A sequence with promoter activity, in accordance with the isolated DNA sequence described immediately above, associated with any other promoter activity (e.g., another promoter sequence, such as a sequence responsive to radiation, hypoxia or tree radicals);

Use of an isolated DNA sequence that comprises the polynucleotide sequence SEQ ID NO:1, which corresponds to a region of the human A33 gene promoter from base pair −105 to base pair +307 or a fragment or variant of this polynucleotide sequence that has been modified by the insertion, substitution or deletion of one or more nucleotides, which has a substantially equivalent function, in order to control the expression of at least one heterologous gene of interest, operably linked to this;

A construct of recombinant DNA expression that includes:

(a) a DNA sequence with promoter activity in accordance with the DNA sequence described above (e.g., apromoter sequence is specific to the control of the expression of the heterologous gene in tumor cells) and

(b) at least one heterologous gene operably linked to this promoter sequence,

where the promoter sequence controls the expression of this, at least, one heterologous gene (e.g., a therapeutic gene);

Constructs as described herein, where the tumor cells are colon tumor cells;

Constructs as described herein, where the heterologous gene is selected from the group of: the E1A gene, a suicide gene such as the hsv-TK gene, the E3 adenoviral genome region, the gene of an interleukin such as IL-10, IL-12 or IL-23;

Constructs as described herein, where the DNA sequence with promoter activity also includes or is found to be associated with any other promoter sequence (e.g., a sequence responsive to radiation, hypoxia or free radicals);

A vector that includes the constructs described above (e.g., a plasmid or viral vector (e.g., a recombinant adenovirus or an oncolytic conditionally replicative adenovirus;

A vector that includes:

(i) a DNA sequence with promoter activity, that comprises the polynucleotide sequence SEQ ID NO 1, corresponding to the region of the human A33 gene promoter from base pair −105 to base pair +307 or a fragment or variant of this polynucleotide sequence that has been modified by the insertion, substitution or deletion of one or more nucleotides, which has a substantially equivalent function; and

(ii) at least one heterologous gene operably linked to the DMA sequence with promoter activity, where the promoter sequence controls the expression of this, at least, one heterologous gene (e.g., a therapeutic gene);

A method of expressing foreign DNA in a host cell, characterized by introducing into the host cell a construct of recombinant DNA expression in accordance with any of claims 6 to 12, where this construct comprises a DNA sequence with promoter activity that includes the polynucleotide sequence SEQ ID NO: 1, corresponding to a region of the human A33 gene promoter from base pair −105 to base pair +307 or a fragment or variant of this polynucleotide sequence that has been modified by the insertion, substitution or deletion of one or more nucleotides, which has a substantially equivalent function; and a heterologous gene operably linked to the DNA sequence with promoter activity, where the DNA sequence controls the expression of this heterologous gene in this host cell;

A pharmaceutical composition that comprises a vector as described herein; and

A method for treating a colorectal tumor, in a patient suffering from this disease, characterized by administrating to the patient an effective quantity of the pharmaceutical composition described herein.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1B are bar graphs showing A33 assays in LoVo, T84, HT-29, CCD841, A375, SB2, T47D, WI-38, HFL-1, and BAEC cell lines, (A) A33 mRNA expression was analyzed using semi-quantitative RT-PCR. (B) A33 antigen promoter activity was analyzed using dual luciferase reporter analysis.

FIG. 2 (A) is a diagrammatic representation of the non viral pAD-I-A33-E1A shuttle plasmid expression cassette.

FIG. 2 (B) is an immunoblot of E1A from LoVo cells left untreated, or transduced with AV22EL at a multiplicity of infection (MOI) of 1 to 500, or wild type, adenovirus at a MOI of 500.

FIGS. 3A-3C is a diagrammatic summary of the adenovirus cloning strategy.

FIGS. 4A-4B are photographs showing the in vitro cytolytic activity of AV22EL and wild type adenovirus. (A) LoVo, HT29, T84, SB2, A375N, T47D, BAEC, HFL-1, WI-38, and HEP-3B cells were seeded in 24 well dishes, transduced with an MOI ranging from 0 to 1000 of AV22EL or wild type adenovirus, and stained with crystal violet (dark shading). (B-D) CCD841, FHC, and LoVo cells were left untreated, or transduced with wild type adenovirus at a MOI of 100 or AV22EL at a MOI of 0.100 and 500. Images were captured using phase contrast microscopy. (E) Mixed cell populations containing T84 (dark shading) and WI-38 cells (clear) were left untreated or transduced with wild type adenovirus of AV22EL. Images were captured using fluorescent microscopy.

FIG. 5 is a bar graph showing the wild type adenovirus or AV22EL titres in CCD841, LoVo, T84, HT29, A375N, SB2, T47D, BAEC, WI-38, and HFL-1 cell lines.

FIGS. 6A-6B are photographs showing the size of multicellular spheroids either left untreated or transduced with wild type adenovirus or AV22EL at MOIs of 10, 100, and 500. (A) Multicellular spheroids comprised A375N cells. (B) Multicellular spheroids comprised LoVo cells.

FIGS. 7A-7B are lines graph showing data collected from in vivo mouse studies using AV22EL. (A) Tumor volume was evaluated over a period of up to 55 days in animals with LoVo or SB2 cell tumors treated with the vehicle control, or transduced with AV22EL. (B) Survival rates were monitored over a period of up to 60 days in animals with LoVo or SB2 cell tumors treated with the vehicle control, or transduced with AV22EL.

FIG. 7C is an image showing representative mice having LoVo cell tumors treated with the vehicle control or transduced with AV22EL.

FIGS. 8A-8B are photographs showing LoVo cell metastatic liver nodules. (A) Animals treated with PBS or transduced with β-galactosidase encoding adenovirus (Ad-βgal) or AV22EL were sacrificed and their livers removed and photographed. (B) Isolated livers were stained with hematoxylin-eosin and evaluated using light microscopy.)

FIGS. 9A-9D are bar graphs showing MTT cell viability assay results in cells transduced with AV22EL or treated with 5-FU either as a monotherapy or a combination therapy, (A and C) LoVo cells. (B and D) HT-29 cells.

### DETAILED DESCRIPTION

Cancer has come to be understood as a class of diseases or disorders characterized by unregulated cellular proliferation. The unregulated, dividing cells can invade immediately adjacent tissue or metastasize to distant sites. The underlying cause is believed to be DNA damage that affects the expression of a specific subset of genes that encode proteins involved in the regulation of cell growth and differentiation. The genes that, when mutated or overexpressed, cause cancer are known as proto-oncogenes, and the mutant gene per se is an oncogene.

Colon cancer, which is also known as colorectal cancer, intestinal cancer, or bowel cancer, can include cancerous growths in the colon, rectum or appendix that originate from the epithelial cells lining the gastrointestinal tract. Polyps are potentially cancerous abnormal growths of tissue that project from a mucous membrane in the colon and can also occur in the stomach, nose, urinary bladder, cervix, small intestine or the uterus. If the polyp is attached to the mucous membrane surface it is said to pedunculated. Conversely, if no stalk is present, the polyp is said to be sessile. An adenoma is another potentially cancerous growth within the colon that is of glandular origin. Adenomas can also arise in the adrenal glands, the pituitary gland, and the thyroid gland. Adenomas are normally initially benign, but over time they may progress to become malignant, and at that point are referred to as adenocarcinomas. Adenocarcinomas are the most common colon cancers, accounting for approximately 90% of all cases. The methods of treating a patient, as described herein, can include a step of identifying a patient (e.g., a human patient) as a candidate for treatment, and that step can include assessing the patient for abnormal growths, including polyps and adenomas in the colon as well, as carrying out any other standard diagnostic or cancer screen.

The treatment currently recommended for colorectal cancer is dependent on the stage of the disease. Currently, surgery is the primary course of action and includes surgeries described as, for example, curative, palliative, bypass, fecal diversion, and “open-and-close.” Approximately 70% of patients affected by colon cancer undergo surgical resection. Unfortunately, approximately 30-40% of these patients subsequently develop recurrent disease. In addition, approximately 10% of patients present a locally advanced disease that is not amenable to surgical resection (Taylor et al., Ann. Surg. Oncol. 9:177-185, 2002; Curley et al., Am. J. Surg. 163:553-559, 1992). These patients have a dismal prognosis and their treatment typically involves combination therapy that can include chemotherapy, radiotherapy, and immunotherapy (Scott et al., Cancer Res. 11:4810-4817, 2005). The methods of treating a patient, as described herein, can be carried out in combination with other therapies, including any of the types just mentioned and specific treatments described further below. For example, a patient receiving an expression vector (e.g., an adenoviral construct) for colorectal cancer can also be treated with any presently known or later developed method for treating colorectal cancer, in addition to surgical intervention, present treatment regimes for colorectal cancer include adjuvant chemotherapy, neo-adjuvant chemotherapy, palliative chemotherapy, immunotherapy, and vaccines. Adjuvant chemotherapeutic agents include 5-fluorouracil (5-FU), capecitabine (Xeloda™), leucovorin (LV; folinic acid), and oxaliplatin (Eloxatin™).

The liver is the most common site of metastatic colon cancer, and the status of this organ, is an important determinant of survival in patients with advanced disease. Complete surgical resection of hepatic metastases can provide a long-term cure for some patients, hut the majority of liver metastases are not tractable to surgery (Mayer-Kuckuk et al., Mol. Ther. 5:492-500, 2002). The present methods of treatment can include a step of assessing a patient to determine whether a colorectal cancer, whether before or after initial treatment, has metastasized to the liver, and the present compositions, when configured for treating colorectal cancer, may do so, at least in part, by reducing the risk that the cancer will metastasize to another location, such as the liver.

Regional chemotherapy delivers tumoricidal agents in a selective fashion, and this can minimize systemic toxicity and damage to normal liver cells. The present compositions can be administered regionally rather than systemically. Chemotherapeutic drugs that are delivered through the hepatic artery can reduce the recurrence of liver metastases after curative resection, and they may also prolong survival when given to patients with unresectable disease. The present pharmaceutical compositions can be administered through the hepatic artery. Immunotherapies with agents such as cetuximab and bevaciozumab, which are antibody-based therapies that target EGF receptor or soluble VEGF, have been used to treat liver metastases, hut the effectiveness of these treatments is contentious because only a short increase in patient survival has been observed (Alekshun et al., Cancer Control 12:105-110, 2005). Another antibody-based therapy, the administration of gefitinib (Irressa™), also tailed to perform as well as expected as a monotherapy for patients with colorectal metastatic cancer (Rothenberg et al., Proc. Annu. Meeting Am. Soc. Clin. Oncol., 2004). It therefore appears that the currently available treatments for colon cancer and metastatic colon cancer are largely ineffective and additional or alternative strategies are likely to be required to significantly increase the rate of patient survival.

Toward that end, DNA sequences and, more generally, nucleic acid sequences are described herein. These sequences can be incorporated in an expression vector, which we may also refer to as a recombinant expression construct. The DNA sequences have been designed to be effective in controlling the transcription of a selected coding sequence, and for that reason, we may refer to them as regulatory sequences. They may be within a 5′ untranslated region of a gene or within an intron (e.g., the intron preceding the first exon). Regulatory sequences are known to include promoters and enhancers, either or both of which may include particular regulatory elements. The present sequences include a DMA sequence mat corresponds to the human A33 promoter, and the nucleic acid sequences can include: (a) a DNA sequence that corresponds to the human A33 promoter; and (b) a coding sequence of a heterologous gene of interest. Sequences that correspond to the human A33 promoter include those having all or a portion of an A33 promoter and all or a portion of the A33 first intron. Where both a regulatory sequence (e.g., SEQ ID NO:1) and a sequence including a coding sequence of a heterologous gene are present, the regulatory sequence can be operably linked to the coding sequence so the heterologous gene of interest is transcribed and translated in a host cell (e.g., a cell maintained in tissue culture or a cell in vivo). Useful DNA sequences that correspond to the human A33 promoter include nucleotides from base pair −105 to base pair +307 of a human A33 (SEQ ID NO:1) and biologically active variants thereof.

In some embodiments, SEQ ID NO:1, which, as noted elsewhere herein, corresponds to a human A33 gene regulatory sequence, can control the expression of at least one heterologous gene (e.g., a single heterologous gene). In other embodiments, SEQ ID NO:1 can be operably linked to a fusion protein including the heterologous gene of interest or, in other embodiments, independently to two or more genes of interest (E1A and other heterologous genes of interest are described further below). In any of these embodiments, SEQ ID NO:1 can also be used together with another regulatory sequence, such as a regulatory or response element that is responsive to a stressor, such as irradiation, hypoxia, heat, and the like. These are defined DNA sequences, which are usually located upstream of the promoter. For example, the present sequences can include an A33 regulatory sequence (e.g., SEQ ID NO:1) or a biologically active variant thereof and a promoter normally associated with a stress protein and/or a hypoxia response element (HRE) or any other element responsive to reactive oxygen species (e.g., the −80 bp to −50 hp of the human VEGF promoter). A CArG motif, as is present in the Egr-1 promoter can also be included (Greco et al., Cancer Gene Ther. 12(7):655-662, 2005). HREs enhance the transcriptional activity of a promoter or responsive element in conditions of low oxygen tension. Hernandez-Alcoceba and collaborators have used HREs to enhance the response of a promoter containing estrogen responsive elements in breast tumors (Hernandez-Alcoceba et al., Cancer Gene Ther, 8:298-307, 2001). These same elements have been combined with radiation response elements (Greco et al., Gene Ther. 9:1403-1411, 2002), which can also be included in the present sequences. These elements can form part of a viral vector (e.g., a replicative or non-replicative adenovirus; Ido et al., Cancer Res. 61:3016-3021, 2001; Park et al., J. Clin. Invest. 110:403-410, 2002; Cowen et al., Cancer Res. 64:1396-1402, 2004). Another useful regulatory element is an NF-κB response element.

The nucleotide sequence of the regulatory sequence and/or regulatory element(s) can be identical to those found in a human. Thus, in one embodiment, the heterologous promoter is human, including, for example, a human A33 promoter or a biologically active variant thereof. Corresponding A33 sequences from other species (e.g., from a non-human primate, or a canine, feline, murine (or other rodent), bovine, or porcine A33 sequence) can also be used as described, herein.

In any of the embodiments described herein, the nucleotide “t” at position 44 of SEQ ID NO:1 can be replaced with die nucleotide “c”.

The A33 antigen is a member of a subfamily within, the immunoglobulin superfamily that includes: (1) the marker of cortical thymocytes in Xenopus (CTX); (2) its chicken ortholog, designated ChT1; (3) mouse and human homologs of CTX; and (4), the receptor for group B Coxsackie viruses and adenoviruses types 2 and 5 (CAR) ((1) Zhan et al., Cancer Gene Ther. 12:19-25, 2005); Shirakawa et al., Clin. Cancer Res. 10:4342-4348, 2004; Sakamoto et al., Cancer Chemother. Pharmacol. 46:Supplemental material 27-32, 2000; (4) Welt et al., J. Clin. Oncol. 8:1894-1906, 1990; and Welt et al., Clin. Cancer Res., 9:1338-1346, 2003.

As noted, variants of SEQ ID NO:1 that are biologically active can also be used to selectively deliver diagnostic or therapeutic proteins to colorectal cancer cells, gastric cancer cells, and any other cell or cancerous cell that naturally expresses an A33 antigen. A biologically active variant of an A33 regulatory sequence is one that drives the expression of a heterologous gene to which it is operably linked to any useful extent. The expression of the heterologous gene may be more or less robust than when SEQ ID NO:1 is used; all that is required is that the expression of the heterologous gene be sufficiently high that it is detectable (in the event the heterologous gene product is assessed in a diagnostic assay) or confers a benefit on a patient (in the event the heterologous gene product is a therapeutic agent).

In particular embodiments, the sequence of the biologically active variant of SEQ ID NO:1 can be at least 30% identical to SEQ ID NO:1 (e.g., at least 35%, 40%, 45%, 50%, 55%, 60%, 65%, 70%, 75%, 80%, 85%, 90%, 95%, 97%, 98% or 99% identical to SEQ ID NO:1), with the differences accounted for by virtue of one or more additions, deletions, or substitutions (or combinations thereof) of a nucleotide present in SEQ ID NO:1. The nucleotides that differ from SEQ ID NO:1 can be found at either end of the sequence or throughout the sequence. For example, in particular embodiments, the sequence of the first 100-105 nucleotides of a biologically active variant of SEQ ID NO:1 can be identical to the first 100-105 nucleotides of SEQ ID NO:1 and the sequence of the remaining nucleotides (nucleotides 100-412) can be absent or can differ from the corresponding nucleotides of SEQ ID NO:1 to the extent noted above (e.g., at least 30%, 35%, 40%, etc. . . . ). Alternatively, the sequence of the first 100-105 nucleotides of a biologically active variant of SEQ ID NO:1 can vary as described above, and the sequence of the remaining nucleotides can be identical to nucleotides 100-412 or 105-412 of SEQ ID NO:1.

The heterologous gene operatively linked to the regulatory sequence can be a full length gene encoding a naturally occurring full length protein, or a truncated or otherwise mutant gene that encodes a truncated or mutant protein. We may use the terms “protein,” “polypeptide,” and “peptide” interchangeably to refer to any amino acid sequence. We recognize that peptides may generally be considered to be shorter than proteins.

A33 is found in about 95% of primary and metastatic colon cancers and in about 38% of diffuse gastric cancers with uniform antigen expression. It has not, however, been observed in most other normal tissues or in other epithelial cancers, sarcomas, neuroectodermal tumors, or lymphoid neoplasms (King et al., Br. J. Cancer 72:1364-1372, 1995). We have described the utility of the present sequences and constructs primarily in 3 the context of treating colorectal cancers. One of ordinary skill in the art would readily appreciate that the present compositions can be used wherever A33 is expressed (as an A33 promoter would be activated in that circumstance); an A33 promoter could drive the expression of a heterologous protein (e.g., an anti-cancer protein) in, for example, an A33-ex pressing cancer cell.

A33 expression is minimal at the base of the crypt in normal intestinal mucosa. This is a characteristic relevant to determining the potential use of A33 antigen as a target for the treatment of colon cancer because the base of the crypt is the site for colonocyte replication. This observation suggests that normal replicating colonocytes will not be targeted by an anti-cancer therapy directed towards an A33 promoter target. A33 may be the first example of a constitutively expressed, tissue-specific epithelial membrane antigen permitting highly cancer-specific targeting in patients with gastrointestinal cancer (King et al., Br. J. Cancer 72:1364-1372, 1995). This observation is further supported by a recent pharmacokinetic study (Scott et al., Clin. Cancer Res. 11:4810-4817, 2005). Consequently, clinical trials have been initialed to target. A33 using antibody based immunotherapy (Welt et al., J. Clin. Oncol. 8:1894-1906, 1990; Welt et al., Clin. Cancer Res. 9:1.338-1346, 2003; and King et al., Br. J. Cancer 72:1364-1372, 1995; see also Welt et al., J. Clin. Oncol. 8:1894-1906, 1990 and Welt et al., J. Clin. Oncol. 12:1561-1571, 1994). Monoclonal antibody A33 (mAbA33) binds to the A33 antigen, which, as noted, is differentially expressed on the surface of some cells, including colon cancer cells.

A33 antigen belongs to the same immunoglobulin superfamily as the Coxsackie adenoviruses type 2 and 5 receptor (Sakamoto et al., Cancer Chemother. Pharmacol. 46suppl:S27-32, 2000; Bergelson et al. Science 275(5304): 1320-1323, 1997). The promoters of the human A33 gene (Johnstone et al., J. Biol. Chem. 277:34531-34539, 2002) and murine A33 gene (Johnstone et al., Am. J. Physiol Gastrointest. Liver Physiol. 279(3):G500-510, 2000) have been cloned and characterized. The comparison between these promoters shows that, just as observed at gene level, there is a high degree of sequence homology. It was also observed that the human A33 promoter possesses two TATA box consensuses (Johnstone et al., J. Biol. Chem. 277(37):34531-34539, 2002) at bp −12 and −224, which are not present in the murine version. Furthermore, both genes contain a transcription initiator sequence (Inr). The promoter contains sites for transcription factors involved in the specific expression of intestinal epithelium such as “Gut-enriched Kruppel-like factors” (GKLF/KLF4 and IKLF/KLF5) (Mao et al., Oncogene 22:4434-4443, 2003). These elements turn be incorporated into the present expression vectors, including viral vectors using retroviruses, adenoviruses, adeno-associated viruses and herpes simplex virus type 1.

As noted, the present compositions can be used in directing a gene of interest in a colorectal tumor cell in such a way that the protein encoded by the gene is expressed and, acting directly or indirectly on cancer cells, improves the state of the illness. The gene of interest can also have diagnostic value, encoding a reporter or marker protein (e.g., a fluorescent protein (e.g., GFP) or antigen, or an enzyme (e.g., β-galactosidase or chloramphenicol transferase).

In one embodiment, a CRAd or oncolytic vector (oncolytic conditionally replicative adenovirus) is prepared using an adenovirus, which comprises a gene of the E1A protein, regulated by a fragment of the A33 promoter DNA sequence. Advantageously, the CRAds control the expression of E1A in colon tumor cells, causing cellular lysis and elimination by replication of the virus concerned. Advantageously, the CRAds containing an E1A gene, as shown in later examples, have an attenuated lytic activity in normal cells, due to their expression governed by a promoter that is expressed principally in tumor cells. More generally, CRAds have been referred to as “new generation” vectors constructed by modifying the adenoviral genome in such a way as to regulate the expression of the E1A gene with a promoter that becomes active in the tissue or cell type required. A viral vector deleted of alt viral open reading frames has been reported and can be used in the present methods (see Fisher et al., Virology 217; 11-22, 1996). Co-expression of viral IL-10 in the present constructs may inhibit the immune response to adenoviral antigen (see Qin et al., Human Gene Therapy 8:1365-1374, 1997).

The gene of interest can be any diagnostically or therapeutically effective gene. More specifically, a gene of interest can be; an adenoviral gene (e.g. an E1A gene or an E3 region of the adenoviral genome), a suicide gene or a gene encoding a pro-apoptotic protein (e.g., a thymidine kinase such as hsv-TK, Bak, Bax, BiK, SIVA, Par-4, a Bcl-2, or a caspase), a tumor necrosis factor gene, or an interleukin (e.g., IL-10, IT-12 or IL-23). Other genes of interest include tumor suppressor genes such as p53, and p202 and PEA3. Other genes of interest encode an enzyme that metabolizes a pro-drug. Other genes of interest encode a toxic protein (e.g., ricin).

The gene of interest can encode a naturally occurring or wildtype protein, and it may also encode a biologically active variant of that protein. As with the A33 regulatory sequence, the sequence of the gene of interest can vary so long as it encodes a protein that functions in a diagnostic assay or therapy to a useful extent. The gene of interest can vary from its wildtype counterpart by virtue of one or more additions, deletions, or substitutions of a nucleotide. Where a substitution is made, it may or may not alter the amino acid residue encoded. The sequence of a biologically active variant of a gene of interest can be at least 50% identical to a corresponding wild type sequence (e.g., 50%, 55%, 60%, 65%, 70%, 75%, 80%, 85%, 90%, 95%, 97%, 98%, or 99% identical). The corresponding protein may also vary to such an extent. For example, a gene of interest can encode a protein that is at least 50% (e.g., 50%, 55%, 60%, 65%, 70%, 75%, 80%, 85%, 90%, 95%, 97%, 98%, or 99%) identical to a corresponding wildtype protein. The gene of interest can encode a viral protein or proteins of other origins (e.g., a human protein).

E1A was so-named because it is the first viral gene to be transcribed in early region 1A (E1A) following Infection. The primary E1A transcript is processed by differential splicing to yield five distinct messages with sedimentation coefficients of 13S, 12S 11S, 10S, and 9S, and the present compositions can include any sequence transcribed into a primary E1A transcript. The E1A proteins, particularly the major ones of 289R and 243R, regulate transcription of both viral and cellular genes in the infected cell. The structure of the E1A mRNA transcripts and positions of the conserved regions in the E1A proteins are known in the art. A comparison of E1A sequences of various human and simian adenovirus serotypes has identified three regions of conserved amino acid homology. In Ad5, conserved region 1 (CR1) maps between amino acid 40-80, CR2 between amino acids 121-139, and CR3 between residues 140-188 which roughly coincides with the 13S unique region. Where one decides to express a biologically active variant of an E1A gene, information of this nature can inform one's decisions. Evolutionary conservation of these particular sequences suggests that they are critical for E1A function (but by no means limits the possibility that other regions are at least equally important) and therefore should be maintained in variant sequences.

The E1A proteins are proline rich, acidic, and localized in the nucleus. Rapid nuclear localization is mediated by a highly basic pentapeptide signal sequence (Lys-Arg-Pro-Arg-Pro) at the extreme carboxyl terminus of the polypeptides. Accordingly, one may wish to include or retain sequences encoding the E1A signal sequence. The conformational constraints imposed by the high proline content likely limit the formation of substantial secondary structure In the E1A proteins. The extreme heat stability of bacterially produced E1A protein, which retains significant transcriptional activation activity even after boding for five minutes, suggests that either E1A can readily refold to an active conformation, or that E1A can function as a random coil. The surprising ability of E1A protein to tolerate large deletions and insertions without total disruption of its biological activities, has led to the concept that E1A is a series of small modular domains that are relatively independent of surrounding sequences. Any known and active deletion or insertion mutant can be expressed by the present sequences.

Generally, any consensus sequence may be (but need not necessarily be) retained. For example, a potential metal binding domain with a consensus sine finger motif (Cys-Xaa₂-Cys-Xaa₁₃-Cys-Xaa₂-Cys) has been identified within the unique region of the E1A 289R protein. The larger E1A protein does bind a single zinc ion, and as expected the smaller E1A protein that lacks tins region does not. This structure appears essential for transcriptional activation by E1A as substitution of glycine for any of the four cysteines within this motif not only abolishes the ability of E1A to bind Zn²⁺ but also results in a loss of transactivation activity.

Residues within a product encoded by a heterologous gene of interest that are post-translationally modified may also be targeted for retention. For example, where the heterologous gene of interest encodes an E1A, one may wish to retain serine residues that are phosphorylated. Post-translational modification of the E1A products is limited to phosphorylation that occurs at serine residues 89, 132, 219, and possibly 96 and 231. However, the literature has suggested that phosphorylation does little to regulate E1A activity.

Adenovirus E1A is a widely investigated small DNA tumor virus oncoprotein that interacts with the retinoblastoma tumor suppressor protein and its relatives, as well as p300 and CBP transcriptional co-activators to deregulate cell cycle progression, which results in p53 stabilization and ultimately p53-dependent apoptosis. In p53 deficient cells the Bcl-2 family members BAX and BAK promote p53-independent apoptosis. More specifically, E1A may promote BAK-dependent apoptosis though the down-regulation of the BAK regulator MCL-1 and subsequent liberation of pro-apoptotic BAK, which results in Bax-dependent apoptosis. In addition, E1A enhances tumor necrosis factor-alpha. (TNF-α) mediated caspase-8 activation by attenuating c-FLIP-short expression, which sensitizes cells to the cytokine TNF-α and thus promotes p53-independent apoptosis (Cuconati et al., Genes Dev. 17:2922-2932, 2003). Therefore, E1A expression is believed to induce cell proliferation followed by apoptosis through a multitude of p53-dependent and independent pathways. As noted above, the present sequences can include sequences encoding not only the heterologous E1A gene (or a biologically active variant thereof), but also sequences encoding other pro-apoptotic genes. One or more of those in the p53 pathway can be included.

Where desired, one can also employ strategies known in the art to improve the efficiency and specificity of vectors encoding an E1A. For example, when viral vectors are used, one can partially or completely remove genes other than the E1A gene. Alternatively, or in addition, E1A can be mutated. For example, one can remove genes encoding E1B-55 kDa or E1B-19 kDa (see also U.S. Pat. No. 5,677,178. Alternatively, or in addition, some viral genes, including E1A can be overexpressed. Overexpression of the adenovirus death protein (ADP) may enhance viral spread and oncolytic efficiency. Thus, the present sequences and/or vectors can include a wildtype viral genome (e.g., an adenovirus genome) or a manipulated viral genome (e.g., an adenoviral genome in which one or more viral gene products not required for viral replication and cell death promotion in tumor cells may be removed, deleted, mutated or manipulated to improve efficacy).

The present constructs may be referred to as conditionally replicating oncolytic viruses as they exhibit cancer cell or tumor specificity; replicate minimally in normal cells or tissues (and/or are non-toxic to cells or tissues); and are highly efficient with respect to replication and cell killing. Conditionally replicating oncolytic adenoviruses have shown promise in the arena of cancer gene therapy (see Yu et al., Curr. Opin. Mol. Ther. 4:435-43, 2002; Post et al., Oncogene 22:2065-2072, 2003; Li et al., Mol. Cancer. Ther. 2:1003-1009, 2003; and Bauerschmitz et al., Mol. Ther. 14:194-174, 2006). While the compositions described herein are not limited to those that work by any particular mechanism, tumor or malignant cell destruction may occur as a result of cell death, including, for example, programmed cell death (apoptosis) and/or necrotic cell death. The replicating oncolytic adenoviruses should spread to, infect, and destroy neighboring tumor or malignant cells while normal or non-cancerous or non-malignant cells remain unaffected.

In the present oncolytic adenoviruses, an adenoviral E1A gene, or a biologically active variant thereof, can be operably linked to a heterologous promoter (e.g., a promoter that is not normally associated with E1A in the viral genome, hot rather one that is normally associated with A33, a gene that is differentially expressed in a colon cancer cell). Generally, a gene and a promoter are operably linked when the promoter drives the expression of the gene. For example, where a promoter is positioned upstream from the initiation site of a gene coding sequence, and the gene is expressed to a greater extent than it would be in the absence of the promoter, one would say that the promoter is operably linked to the gene and/or that it drives gene expression. In the instant ease, where an E1A gene is expressed under the control of a heterologous promoter, E1A expression will be greater than if no promoter were present and may or may not be greater than if E1A's own promoter were present.

The introduction of nucleic acids (e.g., a DNA sequence) into a host cell may be performed using any vector or construct that is capable of being replicated within a host cell. Suitable vectors for the present methods include plasmids, cosmids, artificial chromosomes (e.g., BACs or YACs), DNA viruses, retroviruses, and isolated nucleotide molecules. Transfer can also be performed using liposomes, and liposomes including the present, nucleic acid sequences are also within the scope of the present invention.

The ease with which viruses transfer their generic material from one cell to another has led to their use as genetic vectors. Generally, there is interest in using viruses, including adenoviruses, in cancer therapy, and many articles are available on this topic (see, e.g., Dobbelstein, Curr. Topics Microbiol. Immunol. 273:291-334, 2004). Ideally, such viruses will lyse an infected tumor cell and/or evoke an enhanced host immune response toward the infected tumor cell. Preferably, the cell destructive response will be targeted to cancer cells, with minimal toxicity to non-malignant or non-cancerous cells or tissue (Ring, J. Gen. Virol. 83:491-502, 2002). We may refer to non-malignant or non-cancerous cells or tissues as “normal” or “healthy” cells or tissue. Oncolytic adenoviruses retain the ability to replicate, which can be beneficial in maintaining an effective dose of the adenoviral constructs (Dobbelstein, supra). Adenoviruses infect a large number of human cell types, including epithelial cells, which can give rise to not only colon cancer, but also many other types of malignancies. Adenoviruses are also relatively easy to grow to high titers, and the creation of virus recombinants is well established. Finally, a large body of basic research already exists for this virus type, facilitating its manipulation. Various approaches for modifying and propagating adenoviruses are known in the art and reviewed in various articles including Dobbelstein (supra).

As noted, one example of DNA viruses that can be employed in the current invention are the adenoviruses. There are more than 40 well known serotypes of hum an adenovirus, with the Ad5 adenovirus being especially preferred as the viral vector in the current invention, although the Ad5 capsid and/or modified fibers, such as the adenovirus type 3 capsid and/or ROD fiber, should not be dismissed.

The construction of the appropriate vectors, including those containing a promoter sequence, a gene of interest, and any other element described herein, can be achieved using standard binding, restriction, and cloning techniques, which are well known in the field. DNA sections or sequences at a specific site are obtained using treatment with appropriate restriction enzymes, under the conditions indicated by the supplier, over a period of approximately 3-16 hours. In general, the results of the restriction can be verified by electrophoretic separation in agarose gels (0.8-1.6%) in TAE solution (40 mM triacetate, 2 mM Na₂EDTA.2H₂O, pH 8.5), utilizing ethyl bromide and viewed with UV light in a trans-illuminator (Ultraviolet products Inc., Upland, Calif.). The ligatures can be carried out using the bacteriophage T4 DNA ligase, following the protocols of the provider (New England Biolabs Inc., Beverly, Mass.). An insert:vector ratio of 1:1 to 3:1 can be used, calculating the ratio between the fragments using the following formula:

$$\frac{[ ng vector \times Kb insert ]}{[ Kb vector ]} \times \frac{[ ratio insert ]}{[ vector ]} = ng insert$$

In the vector construct, it is advantageous to be able to distinguish the vector incorporating foreign DNA from unmodified vectors by a quick test. There are known marker systems that generally comprise a gene whose expression confers an identifiable phenotype to the cells transformed when the cells are grown in an appropriate medium. The β-galactoside gene is for example a gene detectable in clones, exhibiting a blue phenotype on plates with X-gal. Thus, a gene encoding a detectable marker can be incorporated into the present sequences and/or vectors in addition to the gene of interest and any other element described herein.

There is a significant body of literature available to provide guidance on the construction and use of expression vectors, including viral vectors. For example, a general description of adenovirus and papovavirus biology can be found in Virology (eds. Fields and Knipe, Raven Press, New York, N.Y.). Although other adenoviral serotypes may be used, adenovirus type 5 provides a common reference point for the nucleotide numbering convention of viral polynucleotides and amino acid numbering of viral-encoded polypeptides of the E1A viral gene region, as well as for other viral genes. Adenovirus type 2 provides a convenient reference for the numbering convention of the E1b viral gene region, and other viral gene regions. One of ordinary skill, in the art could readily identify corresponding positions in other adenoviral serotypes.

DNA sequences of a number of adenovirus types are available from Genbank™. The adenovirus DNA sequences may be obtained from any of the 41 human adenovirus types currently identified. Various adenovirus strains are available from the American Type Culture Collection (Manassas, Va.) or by request from a number of commercial and academic sources. A heterologous gene of interest may be incorporated Into any adenoviral vector and expressed using standard delivery protocols (e.g., by methods used previously to the CFTR or other genes info the vectors). Hybrid adenovirus-AAV vectors represented by an adenovirus capsid containing selected portions of the adenovirus sequence, 5′ and 3′ AAV ITR sequences flanking the gene of interest and other conventional vector regulatory elements may also be used (see, e.g., Wilson et al., WO 96/135.98). For additional detailed guidance on adenovirus and hybrid adenovirus-AAV technology which may be useful in the present methods one can consult WO 94/2893.8, WO 96713597, WO 96726285, and references cited therein.

As described above, the tumor-specific heterologous promoter that is differentially upregulated in a target cancer cell is incorporated into an oncolytic adenovirus to create a conditionally replicating oncolytic adenovirus as a strategy to control, regulate, drive, transcriptionally regulate, transcriptionally control, of transcriptionally drive the expression or transcription of adenoviral genes to promote (1) cancer cell or tumor specificity; (2) minimal replication and/or toxicity in normal cells or tissue; (3) highly efficient viral replication and killing. Viral replication and/or toxicity in normal cells will be minimal but may occur in 1-20% of infected or exposed cells, and may vary on a patient-to-patient basis. Alternatively, viral replication and/or toxicity may occur in 1-15%; 1-10%; 1-5%; 1-3%; 1%; and less than 1% of infected or exposed cells.

The present sequences and/or expression vectors can be formulated as pharmaceutical compositions that may include, for example, a surfactant, a suitable carrier, or a delivery vehicle. This pharmaceutical composition may then be incorporated or packaged as a part of a kit. This kit may include any delivery devices required for the administration of the pharmaceutical composition and instructions for use.

Methods of making the present sequences and expression vectors are also within the scope of the present invention. The methods can include the steps of: (a) providing a gene of interest (e.g., an adenoviral gene) operably linked to a heterologous promoter that is differentially regulated in a target cancer cell and (b) cloning the gene of interest into an expression vector (e.g., an adenoviral backbone or construct). The heterologous promoter can be amplified using the oligonucleotides represented by SEQ ID NOs:5 and 6. The product of this reaction may comprise a PCR product that is 412 base pairs long and includes an A33 promoter. A distinct reaction may be used to acquire an adenoviral gene or any biologically active equivalent thereof, where the adenoviral gene is the adenoviral E1A gene, and the E1A gene is amplified from HEK 293 cells using isolated oligonucleotides SEQ ID NOs:9 and 10. The PCR product of this reaction may be a 1072 base pair fragment including the coding region of an adenoviral E1A gene. The tumor-specific heterologous promoter that is differentially regulated in a target cancer cell or any biologically active equivalent thereof may be cloned into the adenoviral construct in a position that allows the heterologous promoter to control the expression of the gene of interest. The regulatory sequence may “control” the gene of interest by regulating (e.g., transcriptionally regulating) or driving (e.g., transcriptionally driving) the expression of the gene of interest (e.g., an adenoviral gene). There may be intervening sequence between the regulatory sequence and the gene of interest. Typically, the regulatory sequence will be located upstream from the gene of interest.

The invention also provides methods for expressing foreign DNA in a host cell. The methods can be carried out by introducing into the host cell a DNA sequence or expression vector having a regulatory sequence as described herein and, operably linked thereto, a gene of interest. For expression, the cell should be maintained under conditions suitable for maintaining its viability and supporting expression of the gene of interest (e.g., physiological conditions or the conditions of temperature and humidity typically used to culture cells).

The methods for treating cancer can involve administering to the subject or patient requiring treatment (e.g., a human patient), an anti-cancer construct as described herein (e.g., an adenoviral vector in which an adenoviral gene such as E1A is regulated by a heterologous promoter that is differentially regulated in the target cancer cell (e.g., A33). The subject or patient requiring treatment may have early stage or advanced cancer or may be at risk of developing cancer. The patient or subject may have a benign or malignant tumor. The cancer may be of any type in which A33 is expressed (e.g., colorectal cancer or gastric cancer). The subject or patient may also have a colorectal or gastric cancer that has metastasized (e.g., a cancer accompanied by metastatic liver disease). The subject or patient in all the above examples may be a non-human mammal. The subject or patient in all the above examples may be a human.

The present pharmaceutical compositions may be administered via several routes including, for example, orally, intravenously, intranasally, ophthalmicly, and noncomeally. The compositions may also be administered locally to the stomach or colon through, for example, a gastric tube or colonoscope, or they may be injected into an artery such as the intrahepatic artery.

The present compositions, including pharmaceutical compositions containing adenoviruses, can be formulated for therapeutic and diagnostic administration to a patient, For therapeutic of prophylactic purposes, a sterile composition comprising a pharmacologically effective dose of the vector (e.g., a virus such as an adenovirus) is administered to a human patient or a veterinary non-human patient for the treatment of a neoplastic condition. The composition will include the vector and a pharmaceutically acceptable carrier or excipient. Exemplary aqueous solutions that can be used include, for example, water, buffered water, normal saline, a buffered saline (e.g., phosphate-buffered saline), and 0.3% glycine. These solutions are preferably sterile. The composition may also include pharmaceutically acceptable auxiliary substances as required to approximate physiological conditions, such as pH. pH adjusting and buffering agents include, for example, sodium acetate, sodium chloride, potassium chloride, calcium chloride, and sodium lactate, Excipients that enhance adenoviral infection of cells may also be included. Vectors, including adenoviral vectors may be delivered to neoplastic cells by liposome or immunoliposome delivery.

Effective dosages and schedules for administering the pharmaceutical compositions may be determined empirically, and making such determinations is within the skill of one of ordinary skill in the art. Generally, adenoviruses may be administered at a dose of between approximately 1×10²-10²⁰, 1×10³-10¹⁵, 1×10⁴-10¹⁴, 1×10⁴-10¹², 1×10⁴-10¹⁰, 1×10⁴-10¹⁰, 1×10⁴-10⁸, 1×10⁴-10⁶ particle forming units (PFU) per kilogram of body weight of the patient, or at a multiplicity of infection (MOI) in the range of about 0.001 to 100, 0.001-90, 0.001-80, 0.001-70, 0.001-60, 0.001-50. If administered as a polynucleotide (i.e., not packaged as a virus) about 0.01 g to about 1000 g of an adenoviral vector can be administered. It is understood by those in the art that the dose that must be administered to be effective will vary depending on, for example, the mammal, the type of cancer to be treated, the stage of the cancer, the size of the tumor, the location of the tumor, the extent of cancer growth or metastasis, the biological site or body compartment of the tumor, the strain of the virus, the route of administration, the identity of any other drugs, agents, treatments being administered to the mammal, and the toxicity of the heterologous gene of interest. The compositions may be administered one or more times or in multiple doses. It may also be necessary to administer multiple doses of the compositions over an extended period of time (e.g., daily, weekly, bi-weekly, or bi-monthly over weeks, months, or years). The optimal interval between such multiple doses can be determined empirically. Administration of the compositions should be continued until the health of the patient has improved and may be continued until restored. The compositions may be administered with immunosuppressants or in combination with an immunoadsorption procedure (e.g., immunoapheresis) that removes adenovirus from the blood, to attenuate an unwanted immune response towards the virus.

The virus may be administered to a mammal by injection (e.g., intravenous, intralesional, intraperitoneal, subcutaneous, intramuscular, endoscopic, or intraheptic) either at the tumor site by local or regional injection or systemically (e.g., via the bloodstream). The skilled artisan would also appreciate that the virus may also be administered via multiple modes of administration including but not limited to intranasal, oral, rectal, or topical application.

Any of the present compositions may be administered as a part of a combination therapy with a second therapeutic agent or treatment (such as in connection with a surgical procedure or radiation therapy). Use in combination with pharmaceutical agents considered appropriate for the treatment of colorectal or gastric cancers or that are administered to reduce the risk of metastasis are most preferred.

The present compositions may also be administered as a part of a combination therapy with a histone deacetylase (HDAC) inhibitor. HDAC inhibitors have been demonstrated to upregulate CAR expression and thus facilitate adenoviral entry into cells (Wantanabe et al., Exp. Cell Res. 312:256-265, 2006). For example, the present compositions can be administered with a class I or a class II HDAC inhibitor such as FR901228, suberoylanilide hydroxamic acid (SAHA (e.g., MK0638, and vorinostat) and other hydroxamic acids), suberoyl bis-hydroxamic acid, BML-210, depudecin, HC toxin, nullscript, phenylbutrate, valproic acid, scriptaid, splitomicin, suramin sodium, trichostatin A (TSA), APHA compound 8, apicidin, sodium butyrate, pivaloyloxymethyl butyrate, trapoxin B, chlamydocin, depsipeptide, benzamides (e.g., CI-994 and MS-27-275), MGCD0103, NVP-LAQ-824, CBHA, JNJ16241199, tubacin, A-161906, proxamide, oxamflatin, 3-CI-UCHA, AOE, CHAP31, and CHAP 50.

The present invention comprises a method for predicting a patient's response to the composition comprising an anti-cancer construct, including those described herein. A sample that includes cells can be obtained from the patient (e.g., a biopsy sample) and exposed to a cancer-specific construct (e.g., an adenoviral vector that includes an A33 regulatory sequence and a gene of interest). In a first step, one can determine whether the cell, expresses A33, indicating it is cancerous, by detecting the expression of a reporter or marker gene operably linked to an A33 regulatory sequence. In a second step, one can determine whether a patient is likely to respond to a treatment with a therapeutic gene by exposing a sample of cells to a therapeutic construct (e.g., an adenoviral vector in which an A33 regulatory region drives the expression of an E1A gene) and determining whether the rate at which the cells can proliferate is reduced; whether the cells die; or whether the cells have reduced motility. The methods may be facilitated by techniques such as RT-PCR, microarray, immunoblot genetic reporter assay, luciferase assay, blood tests, and green fluorescent protein encoding constructs.

Any of the sequences or vectors described herein can be tested in cell culture assays or in accepted animal models of disease. For example, nude mice can be used to examine the effect of a given sequence and/or vector on a colorectal or gastric cancer.

The constructs or vectors of the present invention can be administered to a patient when needed by injection, oral administration or topically, transported by the appropriate carrier. The appropriate carriers can be aqueous, lipid, liposomal, and the like.

Analyses of variance (ANOVA) were used followed by Tukey's test for the analysis of the data from the luciferase and spheroid assays, and the in vivo experiments. A P value of less than 0.05 was considered to be significant. Similarly, the survival curves were produced according to the Kaplan-Meyer method and the statistical comparisons between the different groups were performed by the application of the log-rank test.

### EXAMPLES

### Example 1

### A33 Antigen mRNA Expression Levels and Promoter Activity

A33 antigen mRNA expression levels were analyzed in 10 distinct malignant or non-malignant mammalian cell lines using semi-quantitative real time polymerase chain reaction (RT-PCR).

The malignant cell lines included three human colon cancer cell lines (LoVo (American Type Culture Collection (ATCC) Accession Number CCL-229), T84 (ATCC Accession Number CCL-248), and HT29 (ATCC Accession Number HTBL-38)), two human melanoma cell lines (A375N (derived from A375 (ATCC Accession Number CRL-1619)) and SB2), and one human breast cancer cell line (T47D (ATCC Accession Number HTB-133)). The non-malignant cell lines analyzed included one human colon cell line (CCD841 (ATCC Accession Number CRT-1790)), two human fetal lung fibroblast cell lines (WI-38 (ATCC Accession Number CCL-75) and HFL-1 (ATCC Accession Number CCL-153)), and one bovine endothelial cell line (BAEC (ATCC Accession Number CRL-1395)).

LoVo, HT29, and T84 were cultured in DMEM/F12 (1:1) (Invitrogen Corp, Carlsbad, Calif.) supplemented with 10% Fetal Bovine Serum (FBS): 2.5 U/ml penicillin; and 2.5 U/ml streptomycin. A375N and SB2 were cultured in MEL medium containing, DMEM (5 g); F12 (5.35 g); NaHCO₃ (2.425 g); ascorbic acid (17.6 g); piruvic acid (150 mg); galactose (300 mg); glutamine (292 mg); streptomycine (132.5 mg); penicillin (63.5 mg); insulin 10 mg/ml (0.5 ml); selenite 35 μg/ml (100 μl); 10% Fetal Bovine Serum (FBS); 2.5 U/ml penicillin; and 2.5 U/ml streptomycin. WI-38 and HFL-1 were cultured in high-glucose DMEM (Invitrogen) with 10% FBS. BAEC was cultured in high-glucose DMEM with 5% FBS. T47D was cultured DMEM/F12 supplemented with bovine insulin (Sigma-Aldrich Corp., St. Louis), CCD841 was cultured in medium containing DMEM (5 g); F12 (5.35 g); NaHCO3 (1.5 g); ascorbic acid (17.6 g); piruvic acid (150 mg); galactose (300 mg); glutamine (292 mg); streptomycine (132.5 mg); penicillin (63.5 mg); insulin 10 mg/ml (0.5 ml); selenite 35 μg/ml (100 μl); triiodotyronine (100 pM); O-phosphoethanolamine (0.01 mM); ethanolamine (0.01 mM); EGF (1 ng/ml); bovine albumin (0.5 g/100 ml); transferring (0.0.1 mg/ml).

RT-PCR reactions were performed using standard procedures. Briefly, 5 μg of RNA was isolated, using Tri-reagent (Sigma-Aldrich), and reverse transcribed using Superscript II reverse transcriptase (Invitrogen) to create complementary DNA (cDNA). This cDNA was then utilized as a template and oligonucleotide primers were used to amplify A33 antigen promoter and GAPDH e-DNA via polymerase chain reaction (PCR). A33 antigen PCR oligonucleotides included SEQ ID NO:2 and SEQ ID NO:3:

| | |
|---|---|
| 5′-CCTGTCTGGAGGCTGCCAGT-3′ | (SEQ ID NO: 2) |
| 5′-AGGTGCATGGGCAGGGTGACA-3′ | (SEQ ID NO: 3) |

GAPDH PCR oligonucleotides included SEQ ID NO: 4 and SEQ ID NO: 5:

| | |
|---|---|
| 5′-ACCACAGTCCATGCCATCAC-3′ | (SEQ ID NO: 4) |
| 5′-TCCACCACCCTGTTGCTGTA-3′ | (SEQ ID NO: 5) |

SEQ ID NOs: 2 and 3 were combined to amplify A33 antigen. SEQ ID NOs: 4 and 5 were combined to amplify GAPDH. PCR was performed using an initial denaturation step (94° C. for 90 seconds) followed by 30 cycles of denaturation (94° C. for 30 seconds), annealing (60° C. for 30 seconds), and extension (72° C. for 30 seconds).

A33 antigen mRNA expression levels were calculated from the mean of three independent semi-quantitative RT-PCR experiments, and are represented herein normalized against the control, GAPDH.

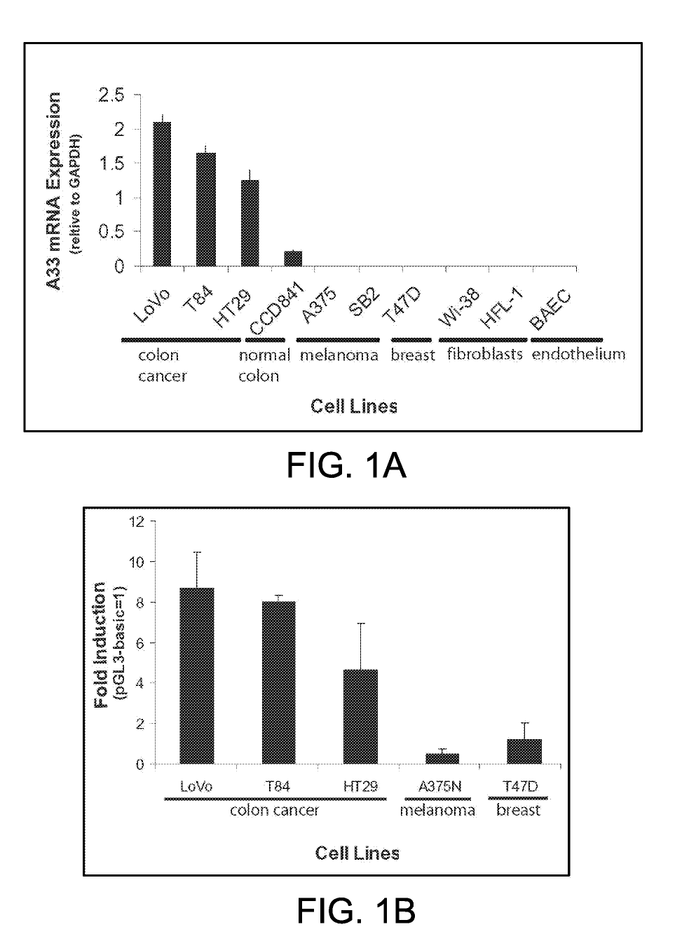

As shown in FIG. 1A, varying levels of A33 antigen mRNA were detected in each of the three human colon cancer cell lines analyzed (LoVo, T84, and HT29). A33 antigen mRNA was also detected in the normal human colon CCD841 cell line, although at levels 9 times lower than those observed in LoVo. A33 antigen mRNA expression was negligible in each of the other malignant and normal cell lines analyzed.

These results suggest that A33 antigen mRNA is exclusively expressed in colon cells, and is highly up regulated in malignant colon cells.

To support the above observation using a technique other than RT-PCR, A33 promoter transcriptional activity was analyzed using dual luciferase genetic reporter assays, as follows.

A genetic reporter construct containing a modified coding region for firefly luciferase (pGL3; Promega) under the transcriptional control of a A33 antigen promoter (A33Pr), herein designated SEQ ID NO:1, was generated using molecular biology techniques that are well known in the art. Briefly, a 412 base pair (bp) fragment, comprising nucleotides −105 to +307 relative to the transcription start codon in the published A33 antigen promoter sequence (AF200626) was amplified from a human lymphocyte genomic DNA using oligonucleotides SEQ ID NO: 6 and SEQ ID NO: 7, shown below (lower case font denotes a restriction endonuclease site):

(SEQ ID NO: 6)

5′-GGctcgagCAGCAAATATGGGCAACACCC-3′

(SEQ ID NO: 7)

5′-GGGCagatctGCACTGGCAGCCTCCATACAGG-3′

SEQ ID NOs: 6 and 7 were combined and A33Pr amplified using standard PCR. The A33Pr PCR product was then digested using XhoI and BglII restriction endonucleases (RE), which recognize the RE sites in SEQ ID NOs: 6 and 7, respectively, as indicated above, and cloned into pGEM-Teasy (Promega Corp. Madison, Wis.) to create a construct designated pGEM-A33. Cloning was confirmed by sequence analysis using the universal primers SP6 (SEQ ID NO:8) and T7 (SEQ ID NO:9) and is shown as SEQ ID NO:10, wherein A33Pr starts at nucleotide 49 and continues to nucleotide 460.

A33Pr was subsequently sub cloned from pGEM-A33 into pGL3 using the BglII and XhoI RE sites located immediately upstream from the modified luciferase coding region and the resulting luciferase reporter construct was designated pGL3-A33. Cloning was continued by sequence analysis using the universal primers P2 (SEQ ID NO: 11) and p3 (SEQ ID NO: 12) and is shown as SEQ ID NO: 13; wherein A33Pr starts at nucleotide 97 and continues to nucleotide 508.

Reporter assays were performed by transfecting pGL3-A33 into human colon cancer (LoVo, T84, and HT29), melanoma (A375N), and breast cancer (T47D) cell lines. Briefly, cells were seeded at a density of 4×10⁴ cells per well in 24-well plates and incubated for 24 hours. 0.8 μg pGL3-A33 and 0.1 μg of the control reporter pRL-CMV (Promega Corp. Madison, Wis.) were then transfected into cells using Lipofectamine 2000 according to the manufacturer's instruction (Invitrogen). Alternatively, pGL3-A33 was substituted for the control constructs 0.80 μg pGL3-Basic (Promega Corp. Madison. WI), which contains a luciferase gene but lacks a eukaryotic promoter or enhancer, or pGL3-promoter (Promega Corp. Madison, Wis.), which contains a SV40 promoter upstream of a luciferase gene. Cells were harvested 46 hours post transfection, and dual luciferase assays were performed using a Genios luminometer (TECAN, Maennedorf, Switzerland). Mean data were calculated from three independent experiments, and are represented relative the control plasmid pGL3-Basic, described above.

As shown in FIG. 1B, A33Pr was active in the three colon cancer cells tested (LoVo, T84, and HT29), with the highest activity observed in LoVo cells. Furthermore, A33Pr reporter activity was highly consistent with the A33 mRNA expression levels observed in FIG. 1A. A33Pr activity was not observed in the melanoma (A375N) or breast cancer (T47D) cell lines.

### Example 2

### Adenovirus Construction

Human adenoviral expression systems are widely used due to their potential for gene transfer and protein expression in mammalian cells, and the methods and protocols required for their construction are well known in the art.

Adenovirus construction typically requires cloning the gene of interest into a non-viral shuttle plasmid, and cotransfection of the linearized shuttle plasmid with a linearized adenoviral construct into a packaging cell line, such as HEK 293. Viral recombination and propagation then occurs over a period of 1 to 10 days. The adenovirus developed herein, designated AV22EL, was constructed using such, a standard technique.

The non-viral shuttle plasmid we used was generated by modifying the previously described pAdYPSY shuttle plasmid, which contains the extreme left of human type 5 adenovirus with E1 and E3 regions deleted and replaced with a Rous sarcoma virus (RSV) promoter and SV40 polyadenylation signal (Mariano et al. (2005) Cancer Research. 65: 5123-5132). First, the pADYPSY RSV promoter sequence was replaced with SEQ ID NO: 14, which is a multiple cloning site (MCS), having SpeI, BclI, KpnI, NheI, MluI, BglII, EcoRV, ClaI, SnaBI, and SalI RE sites, to increase the cloning capacity of pADYPSY and obtain a construct that we designated as pAd-XP.

Second, a 234 bp sequence corresponding to the β-globin stop codon coding region was cloned into the pAD-XP MCS to insulate the downstream elements from the viral transcriptional regulatory machinery (Steinwaerder et al. (2004) Human Gene Ther. 15: 995-1002). The β-globin insulator was amplified using oligonucleotides SEQ ID NO: 15 and SEQ ID NO: 16 shown below. Note, lowercase font denotes a restriction endonuclease site.

| | |
|---|---|
| 5′-CCactagtGCTAGAGCTCGCTGATCAGC-3′ | (SEQ ID NO: 15) |
| 5′-CggtaccATCCCCAGCATGCCTGC-3′ | (SEQ ID NO: 16) |

SEQ ID NOs: 15 and 16 were combined to amplify the β-globin insulator and the resulting PCR product was digested using SpeI and KpnI which recognize the sequences indicated by the lower case font in SEQ ID NOs: 15 and 16, respectively, and cloned into the pAd-XP MCS, to obtain pAd-I-XP. Cloning was confirmed by sequence analysis using the primers pAd-sense (SEQ ID NO: 18) and pAd-antisense (SEQ ID NO: 19) and is shown as SEQ ID NO: 17.

Third, the adenoviral E1A gene corresponding to nucleotides 560 to 1632 within the adenoviral genome was amplified using the oligonucleotides SEQ ID NO: 20 and SEQ ID NO: 21:

(SEQ ID NO: 20)

5′-CGAGATCTCCGGGACTGAAAATGAGACAT-3′

(SEQ ID NO: 21)

5′-GCGGATCCAAACATTATCTCACCCTT-3′

SEQ ID NOs: 20 and 21 were combined to amplify E1A. The resulting E1A PCR product was cloned into TOPO-pCR4, and cloning was confirmed by sequence analysis and is shown as SEQ ID NO: 22. E1A was then sub cloned into pcDNA3 (Invitrogen) and E1A protein expression was verified in HEK 293 cells by immunoblotting using M73 anti-E1A antibody (Santa Cruz Biotechnology).

The E1A construct, was then excised from pcDNA3 using BglII and BamHI and cloned into the BglII RE site located in pAd-I-XP and pAd-XP, described above. The E1A construct was cloned downstream of the β-globin insulator cloned in pAD-I-XP. The resulting plasmids were designated pAD-I-XP-E1A. (SEQ ID NO: 23) and pAD-XP-E1A (SEQ ID NO: 24). SEQ ID NO: 23 shows the β-globin insulator (nucleotide 71 to nucleotide 314) and E1A (starting at nucleotide 344).

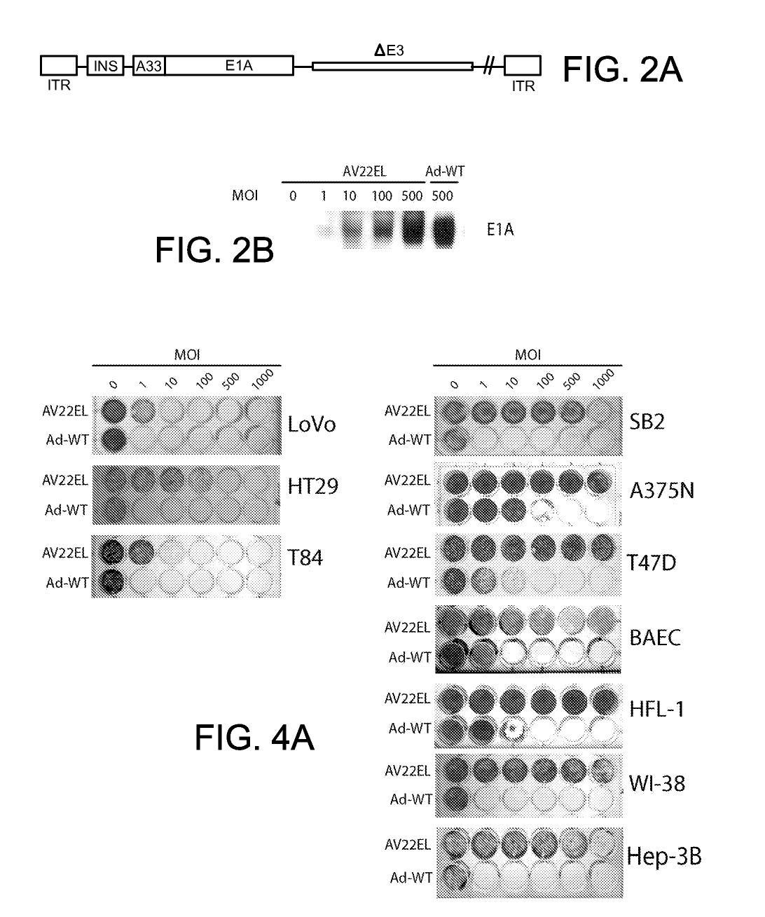

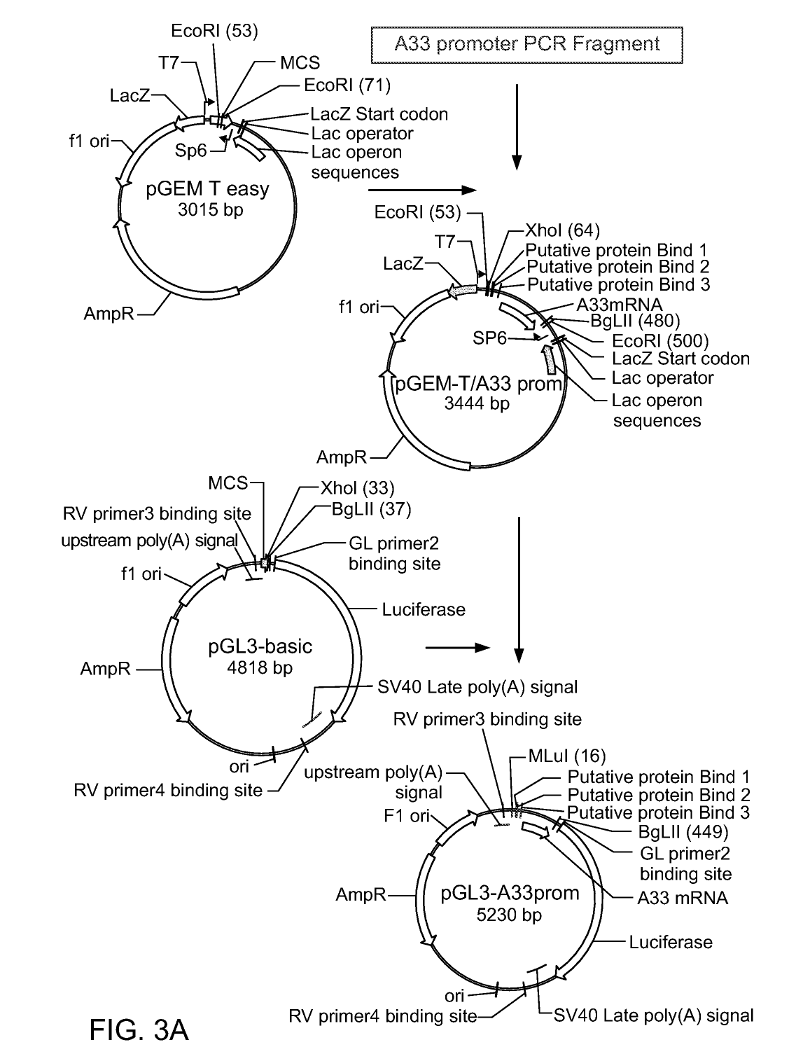

A33Pr SEQ ID NO:1 was then sub cloned into pAD-I-XP-E1A directly from the above described pGEM-A33 (SEQ ID NO: 10). A33Pr was excised from pGEM-A33 (SEQ ID NO: 10) using MluI and BglII and cloned into the pAD-I-E1A MCS immediately upstream from E1A. The expression cassette of the final construct, designated pAD1-A33-E1A is depicted in FIG. 2A. Cloning was confirmed by sequence analysis using pAd-sense (SEQ ID NO: 18) and pAd-antisense (SEQ ID NO: 19) and is shown as SEQ ID NO: 25. SEQ ID NO: 25 shows the β-globin stop codon coding region (nucleotide 72 to nucleotide 314); A33Pr (nucleotide 365 to nucleotide 765) and E1A (starting approximately at nucleotide 766).

pAD-I-A33-E1A (SEQ ID NO; 25) was then linearized using the restriction endonuclease FspI, Adenovirus AV22EL was created by transfecting linearized pAD-T A33-E1A shuttle plasmid into HEK 293 with adenovirus type 5 linearized with ClaI, as previously described by Bett et al. (1994) PNAS. 91: pp. 8802-6. Viable virus was purified as previously described, by Lieber et al. (1996) J. Virol. 70: 8944-60. AV22EL was sequenced using SEQ ID NO: 18 (shown herein as SEQ ID NO: 26) and SEQ ID NO: 19 (shown herein as SEQ ID NO: 27).

AV22EL particle concentrations were determined using OD₂₆₀, and 50% tissue culture infective doses (TC ID₅₀) were determined by performing a standard plaque assay in HEK 293 cells.

AV22EL E1A expression was verified by transducing LoVo cells with an increasing multiplicity of infection (MOI) of virus. Cell lysates were collected 72 hours post infection and E1A protein expression, was confirmed by immunoblotting using M73 antibody.

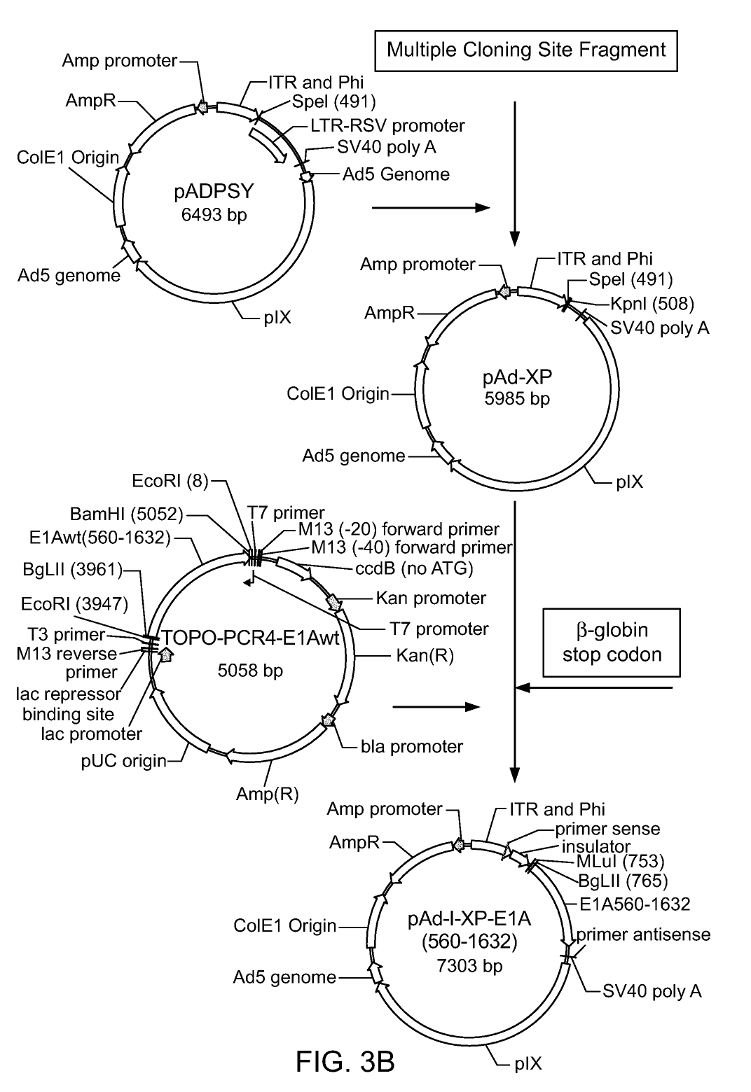

As shown in FIG. 2B, E1A was efficiently expressed in LoVo cells transduced with AV22EL. Furthermore, AV22EL E1A expression levels were comparable to those detected for wild type adenovirus (Ad-WT). A summary of the above described cloning strategy is illustrated in FIG. 3.

### Example 3

### Analysis of AV22EL In Vitro Oncolytic Activity

The oncolytic capability of AV22EL was analyzed using in vitro cytotoxicity studies, as follows. Three human colon cancer cell lines (LoVo, HT29, and T84), two human melanoma cell Hoes (SB2 and A375N), one human breast cancer cell line (T47D), one bovine endothelial cell line (BAEC), two human fetal lung fibroblast cell lines (HFL-1 and WI-38), and one human hepatocellular carcinoma cell line (Hep-3B), were seeded in 24-well plates at a density of 1×10⁴ cells per well. The next day, cells were infected with AV22EL or Ad-WT at a MOI ranging from 1 to 1000. Cells were stained with crystal violet 10 days post infection and photographed.

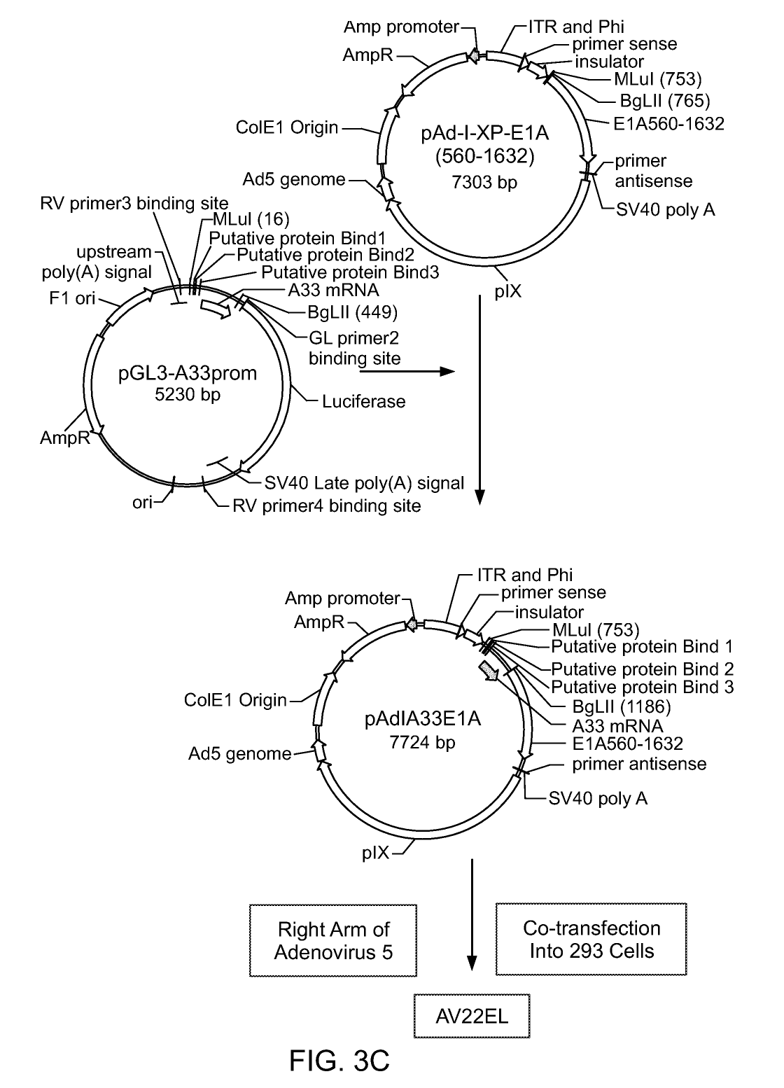

As shown in FIG. 4A, healthy cells are represented by a positive crystal violet stain (dark color). Conversely, infected cells with reduced viability are represented by reduced crystal violet staining (clear wells).

As shown in FIG. 4A, AV22EL was highly effective against each of the three colon cancer cell lines, particularly LoVo and T84. Interestingly, however, AV22EL was at least 2 orders of magnitude less effective against HT29. AV22EL also promoted a cytolytic response in the non-colon cancer cells lines SB2, A375N, and Hep-3B cells, however, only at an MOI of 1000. No effect was observed in T47D, WI-38, HFL-1, or BAEC. In contrast, Ad-WT was effective in most cells at an MOI of 1.

These results indicate that AV22EL is cytolytic in colon cancer cells but not in melanoma, breast cancer, or hepatoma cell lines. In other words, AV22EL is selectively oncolytic in colon cells. Interestingly, the oncolytic capability of AV22EL appears to be proportional to the A33 activity levels described in Example 1. Ad-WT, which does not encode A33Pr, is not selective and is highly cytolytic in all cell lines.

AV22EL was also tested using additional cytotoxicity studies in CCD841 and FHC normal human colon cells, and LoVo colon cancer cells. Cells were seeded as described above, and transduced with AV22EL at a MOI of 100 or 500, or Ad-WT at a MOI of 100. 10 day post infection cells were analyzed for morphological signs of cytotoxicity using phase contrast microscopy.

As shown in FIGS. 4B, C, and D, AV22EL was cytolytic in LoVo colon cancer cells (cells are rounded and clearly dead or dying) but not in the normal colon cell lines tested (cells have regular healthy morpholgies). In contrast, Ad-WT was highly cytolytic in all cell lines.

These results are highly consistent with FIG. 4A, and further confirm the selective oncolytic capability of AV22EL.

AV22EL was also tested in mixed cell populations containing T84 colon cancer cell lines expressing green fluorescent protein (dark shading) and WI-38 human fibroblast cells. As shown in FIG. 4E, AV22EL selectively killed T84 (dark shading top and bottom panel) cells but not WI-38. Interestingly, both cell types were equally permissive to adenoviral infection. In contrast, Ad-WT eliminated both cell types (top panel).

These results suggest that although AV22EL infects all cells in a mixed population, it is oncolytic only against colon cancer cell lines. Together, the results described above unequivocally demonstrate that AV22EL is selectively oncolytic against colon cancer cells.

### Example 4

### AV22EL Replication in Malignant and Normal Cell Lines

As an alternative strategy to confirm the selectivity of AV22EL, viral transcription was analyzed in a selection of malignant and normal cell lines. Briefly, cells were seeded in 6 well plates at a density of 1×10⁵ cells per well. The next day, cells were exposed to either AV22EL or Ad-WT at a MOI of 50 for 2 hours. Virus containing medium was then removed and cells were cultured for 72 hours. Viral titers were then determined, according to Li et al. (2001) Cancer Res. 61: 6428-36.

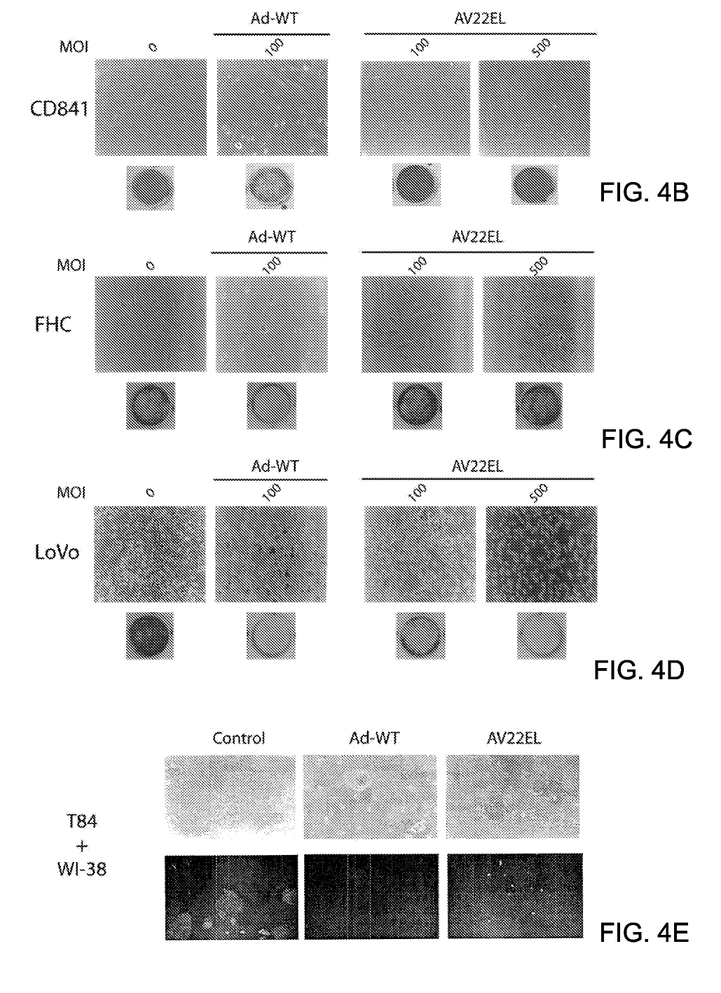

As shown in FIG. 5, AV22EL titers varied significantly in the different cell lines, indicating different degrees of viral replication, with the highest titers observed in the colon cancer LoVo and T84 cell lines. Conversely, AV22EL did not appear to replicate in the normal colon cells, fibroblasts, or endothelial cells. These data are highly consistent with the A33 mRNA levels presented in FIG. 1A. Interestingly, AV22EL titers were comparable in A33 positive colon cancer HT29 cells and A33 negative melanoma SB2 cells refer to Example 1 for quantification of A33 expression levels) due to the fact that SB2 cells are highly permissive to adenoviral infection, as determined using β-galactosidase encoding adenovirus (Ad-βgal). In other words, the viral titers observed for SB2 cells are due to high viral uptake, and not Viral replication. Ad-WT was equally expressed in all cell types tested.

These data further support the selective capacity of AV22EL towards A33 positive colon cancer cells.

### Example 5

### AV22EL Oncolytic Activity In Multicellular Spheroids

Multicellular spheroids mimic the in vivo environment of an avascular tumor and are frequently used as in vitro tumor models. The specificity and oncolytic activity of AV22EL was, therefore, analyzed in multicellular spheroids composed of human melanoma A375N cells or human colon cancer LoVo cells.

Multicellular spheroids were cultured using the semi-solid liquid overlay technique according to Lopez et al. (2006) Mol. Cancer. Ther. 5: 2503-11. Briefly cells were seeded in 96 well plates at a density of 1×10⁴ cells per well, wherein each well contained semi-solid 1% (w/v) agarose in 200 μL of medium. Cells were then cultured for 72 hours, or until spheroids formed. Spheroids were then infected with AV22EL or Ad-WT at a MOI of 10,100, or 500. Spheroids were subsequently photographed and measured 7 days post infection.

TABLE 1
AV22EL Lytic Activity in Multicellular Spheroids

| | No | Ad-WT | | No | AV22EL | |
|---|---|---|---|---|---|---|
| | Virus | (MOI 500) | | virus | (MOI 500) | |
| Cell | Spheroid | | % | Spheroid | | % |
| type | Volume (mm³) | | difference | Volume (mm³) | | difference |
| A375N | 26 | 14.73 | 48 | 21 | 20 | 7 |
| HeLa | 29 | 15 | 48 | 21 | 24 | 0 |
| LoVo | 11 | 4 | 60 | 14 | 5 | 62 |

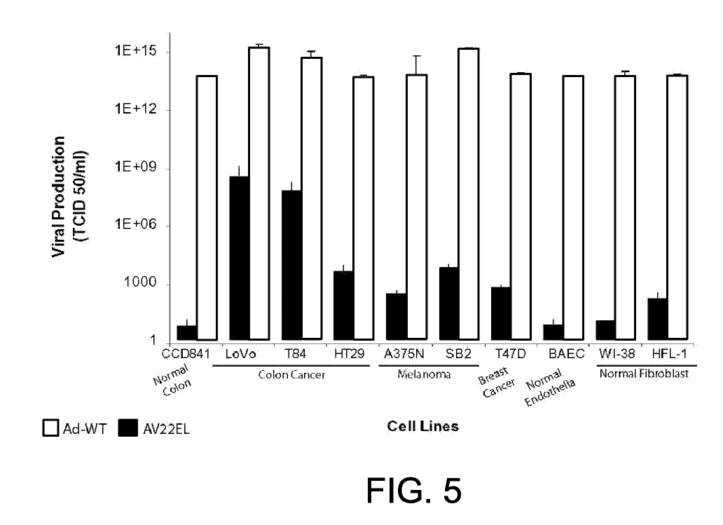

As shown in FIG. 6A and Table 1, respectively, AV22EL did not reduce the size of A375N or HeLa spheroids. In contrast, AV22EL promoted a 62% reduced the size of a LoVo colon cancer cell spheroid. Ad-WT evoked a non-specific reduction in spheroid size for all three cell lines.

These results further confirm the selective oncolytic capacity of AV22EL towards colon cancer cells and demonstrate that the virus is effective against cells in tumor-like environment.

### Example 6

### AV22EL In Vivo Oncolytic Activity

To ascertain the in vivo oncolytic capability of AV22EL, 5 to 6 week old nude mice were xenotransplanted using subcutaneous injection in the flank with tumorigenic inocula consisting either of 5.0×10⁶ human colon cancer LoVo cells or human SB2 melanoma cells. Tumor volumes were then estimated twice a week using caliper measurements, and mice were randomly separated once the volume reached 100 mm³. Groups were then randomly treated with either AV22EL (1×10¹⁰ viral particles per mouse) or vehicle (PBS), which were both administered via intra-tumoral injection at days 1, 4, and 7. Animals were then studied for up to 55 days post infection.

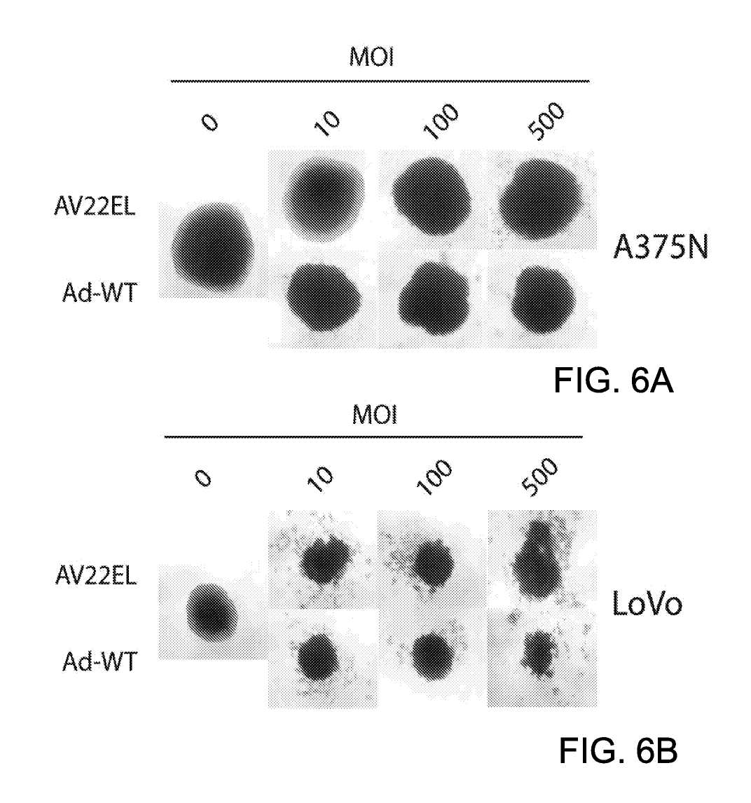

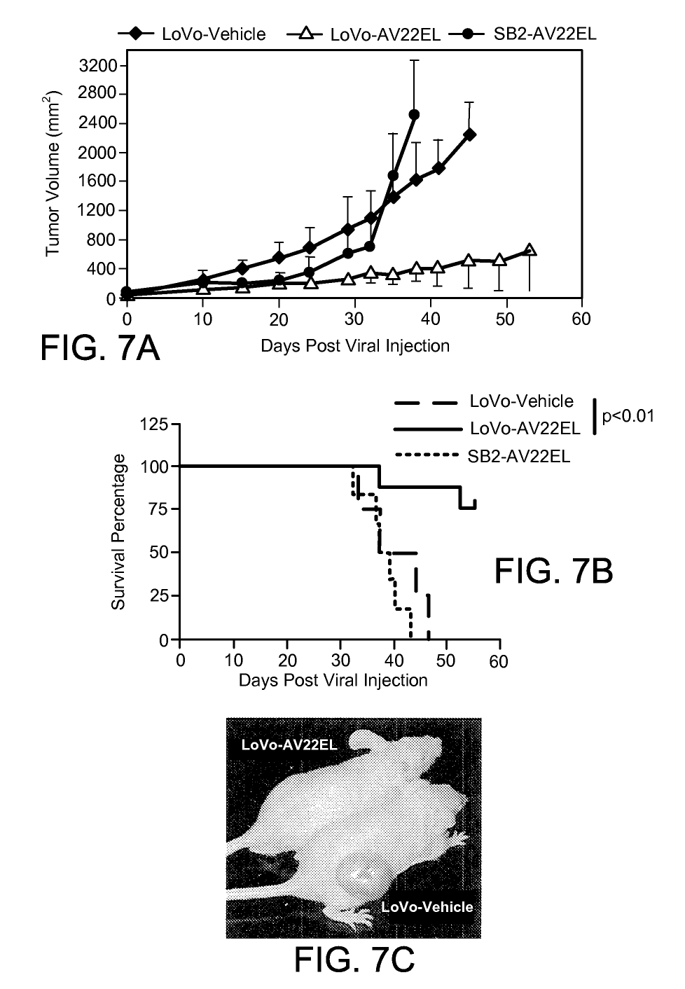

As shown in FIG. 7A, AV22EL reduced the volume of LoVo tumors for the duration of this study. In contrast, AV22EL did not reduce the volume of SB2 tumors, despite the fact that these cells are highly permissive to adenoviral infection (see Example 4). Likewise, no reduction in tumor volume was observed in mice treated with the vehicle control.

As shown in FIG. 7B, in addition to reducing the volume of LoVo tumors, AV22EL also increased the survival rate of the animals, with a 100% survival rate up to day 40, and a 75% survival rate to the end of the study (approximately 55 days). In contrast, AV22EL did not protect SB2 tumor mice, and all animals died before day 43 of this trial. Likewise, all control vehicle animals died before day 43 of this trial. Representative images of AV22EL and vehicle treated mice with LoVo tumor are presented in FIG. 7C. During the course of this study, none of the animals presented any signs of wasting or visible indications of toxicity.

These data support the in vivo application of AV22EL by demonstrating that AV22EL is potently oncolytic against in vivo colon cancer tumors.

### Example 7

### Treatment of Liver Metastasis Using AV22EL

Colon cancer therapy invariably requires the treatment of metastatic disease. Therefore, an in vivo system was developed to promote liver metastases in mice. These animals were then utilized to determine the therapeutic potential of systemic administration of AV22EL against the growth of the established liver metastases.

Liver metastases was induced by injecting human colon cancer LoVo cells into the portal vein of male and female nude mice. 7 days post inoculation, parallel groups of mice were sacrificed and examined for indications of liver metastases. Following confirmation that liver metastases had developed, the remaining live animals were treated with AV22EL, control virus (Ad-βgal), or vehicle (PBS) administered via tail vein injections on days 7, 10, and 14 following the initial LoVo injection. Mice were then sacrificed 7 days later and liver samples were processed using routine histological methods, including hematoxilin-cosin, and evaluated using light microscopy.

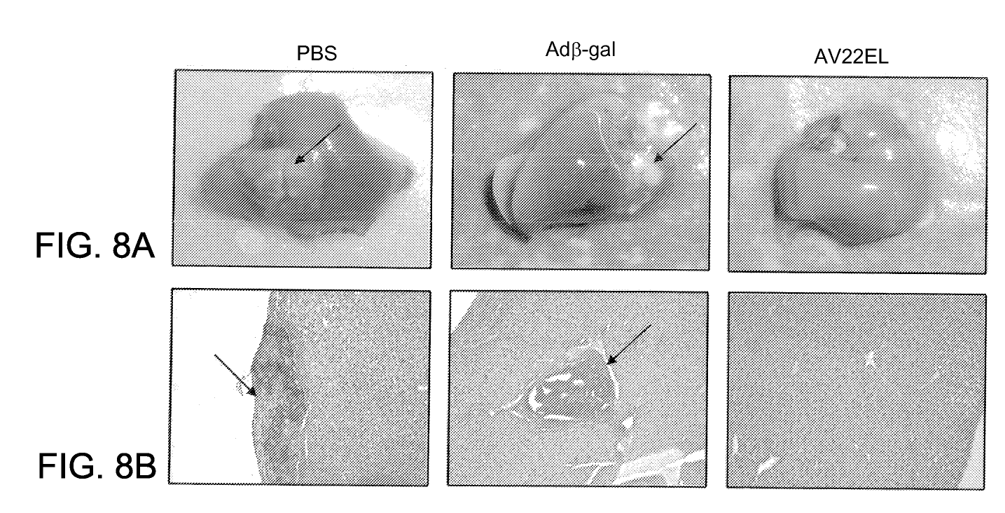

AV22EL reduced the appearance of hepatic metastatic nodules in 10 out of 11 (90%) of treated animals, irrespective of gender. In contrast, treatment with Ad-βgal or vehicle controls reduced the appearance of nodules in 1/10 (10%) and 0/4 (0%) animals, respectively. Representative livers are shown in FIG. 8A. All gross observations were continued using hematoxylin-cosin staining and representative images are shown in FIG. 8B. Parallel Ad-βgal infections and β-galactosidase staining was used to confirm that the administered adenoviruses targeted hepatitic metastases.

These data further support the in vivo application of AV22EL by demonstrating AV22EL therapy is effective at diminishing colon cancer cell metastases, even when administered indirectly.

### Example 8

### AV22EL Toxicity Testing

Prolonged exposure to AV22EL is not associated with any morphological signs of liver toxicity, as shown in FIG. 8B. Nevertheless, standard biochemical tests were performed to assess liver function, in AV22EL and control treated animals. As shown in Table 2, hallmark indications of altered liver function, as characterized by decreased levels of serum albumin with concurrent increases in AST and ALP levels, were detected in untreated, Ad-βgal and vehicle treated metastatic animals. In contrast, animals exposed to AV22EL presented normal serum albumin, ALT, and ALP levels, indicating normal liver function.

This result demonstrate that AV22EL is not only is not hepatotoxic and further support the in vivo application of the virus,

TABLE 1
Biochemical Analysis of Liver Function

| | | Vehicle | | |
|---|---|---|---|---|
| Treatment | None | (PBS) | Ad-βgal | AV22EL |
| Animals with | 0 | 90 | 100 | 9 |
| metastases (%) | | | | |
| Albumin (g/dL) | 3.45 ± 0.07 | 2.66 ± 0.11 | 2.7 ± 0.17 | 3.125 ± 0.3 |
| AST (U/L) | 229 ± 52 | 447 ± 88 | 300 ± 38 | 170 ± 69 |
| ALP (U/L) | 110 ± 16 | 140 ± 37 | 114 ± 18 | 107 ± 35 |

### Example 9

### Combination Therapy Using AV22EL and 5-FU

Combination chemotherapy is a classical approach to improving chemotherapeutic efficacy in cancer patients. Additional in vitro experiments were conducted to explore the usefulness of combining the commonly used chemotherapeutic, 5-FU, and AV22EL as a therapeutic strategy for the treatment of colon cancer.

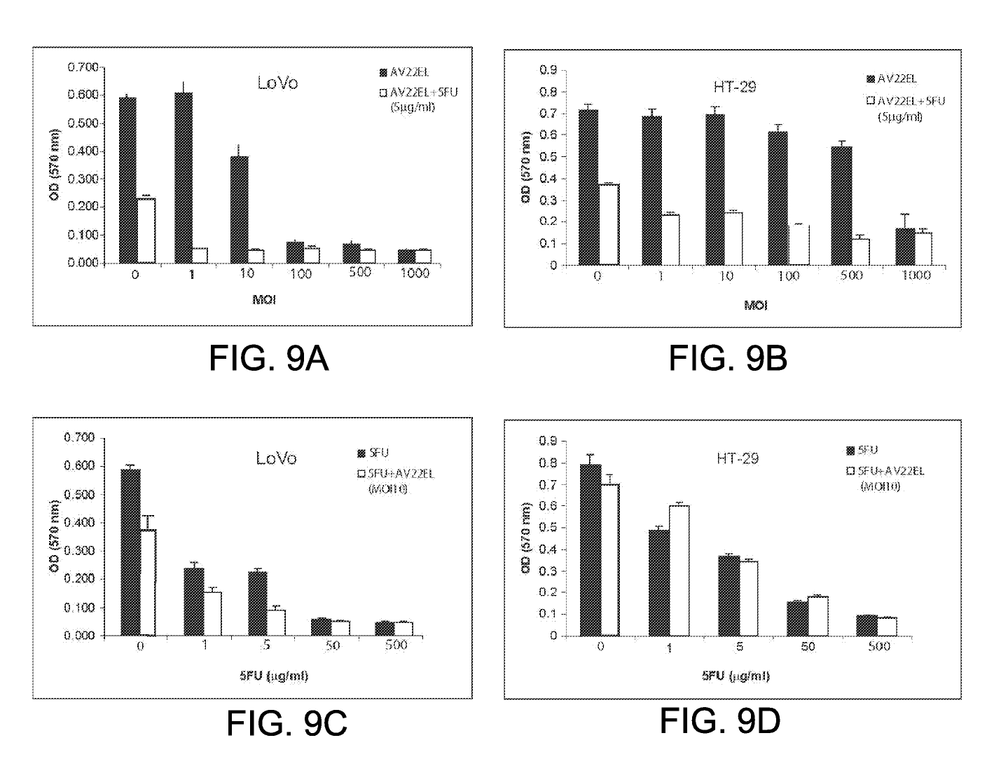

Human colon cancer HT-29 and LoVo cells were seeded in 96 well flat bottomed plates at a density of 2×10³ cells/well. The next day, cells were infected with an increasing MOI of AV22EL alone, or as a pre-treatment in combination with 5 μg/ml 5-FU (e.g., FIGS. 9 A and 9B), as follows. All cells were infected with AV22EL for 24 hours prior to removal of the virus containing medium. Cells requiring combination therapy were then cultured in fresh medium containing 5-FU for an additional 4 days. Alternatively, cells were treated with increasing concentrations of 5-FU alone or as a pre-treatment in combination with AV22EL at a MOI of 10 (e.g. FIGS. 9C and 9D). All cells were treated with 5-FU for 24 hours prior to removal of the 5-FU containing medium. Cells requiring combination therapy were then Infected with AV22EL for 4 days. Cells were analyzed 5 days post treatment using the commercially available colorimetric MTT cell viability assay. Briefly, cells were incubated in 100 μl PBS containing 0.5 mg/ml MTT for 4 hours. The MTT solution was then replaced with DMSO and absorbance was determined at 570 nm using a microplate reader (BIO-RAD). All samples were repeated in 6 independent experiments and data were plotted graphically, as shown in FIGS. 9A, 9B, 9C, and 9D.

As shown in FIGS. 9A and 9B, AV22EL monotherapy (black bars) evoked a cytolytic effect on both LoVo and HT-29 cells. Consistent with FIG. 4A, LoVo cells were more susceptible to a AV22EL than HT-29, with cytolytic effects observed at MOIs of 10 and 500, respectively. As shown in FIGS. 9C and 9D, LoVo and HT-29 are also sensitive to 5-FU monotherapy. Interestingly, however, treatment of LoVo and HT-29 cells with AV22EL prior to their exposure to 5-FU significantly improved the cytolytic effects of the individual therapies. Furthermore, this effect appears to be a synergistic, and not simply additive. Surprisingly, pre-treatment with 5-FU in combination with AV22EL did not improve the cytolytic effect of the individual therapies. In addition, no differential effect was observed using combination therapy in which AV22EL and 5-FU were added simultaneously.

Thus, the A33 antigen promoter confers competence for selective replication in colon cancer cells. These data suggest AV22EL may be effective as an anticancer therapeutic.

A number of embodiments of the invention have been described. Nevertheless, it will be understood that various modifications may be made without departing from the spirit and scope of the invention. For example, one can use another type of naturally occurring viruses that have demonstrated some tumor-selectivity in their replication and cytolysis, including, the minute virus of mice, H1 human reovirus, and vesicular stomatitis virus (VSV). These viruses are referred to by those in the art as oncolytic viruses. Most viruses, however, must be genetically modified in order to exhibit tumor-selectivity, for example, through the introduction of a heterologous promoter. The advantage of this technique is that viral selectivity can be tailored to specific cell types and cancer cells. Accordingly, other embodiments are within the scope of the following claims.

1.-11. (canceled)

12. A nucleic acid sequence comprising a first regulatory sequence comprising SEQ ID NO:1 or a biologically active variant thereof and, operably linked thereto, a heterologous gene of interest.

13. The nucleic acid sequence of claim 12, wherein the biologically active variant comprises a sequence that is at least 50% identical to SEQ ID NO:1.

14.-15. (canceled)

16. The nucleic acid sequence of claim 12, further comprising a second regulatory sequence activated by a stressful stimulus.

17. The nucleic acid sequence of claim 16, wherein the second regulatory sequence comprises a hyposix response element, a reactive oxygen species response element, an NFκB response element, a promoter naturally associated with a stress protein, or a CArG motif.

18. The nucleic acid sequence of claim 12, wherein the heterologous gene of interest encodes a therapeutic gene product and the nucleic acid sequence optionally includes an internal ribosome entry site (IRES).

19. The nucleic acid sequence of claim 18, wherein the therapeutic gene product is an adenoviral protein, a pro-apoptotic protein, a tumor necrosis factor, or an interleukin.

20. The nucleic acid sequence of claim 19, wherein the adenoviral protein is E1A.

21. The nucleic acid sequence of claim 19, wherein the pro-apoptotic protein is Bak, Bax, SIVA, Par-4, a Bcl-2, thymidine kinase, or a caspase and the interleukin is IL-10, IL-12, or IL-23.

22. (canceled)

23. An expression vector comprising the nucleic acid sequence of claim 12.

24.-27. (canceled)

28. An isolated cell comprising the expression vector of claim 23.

29. A pharmaceutical composition comprising the expression vector of claim 23.

30. The pharmaceutical composition of claim 29, wherein the composition is formulated for oral administration, intravenous administration, or intrahepatic artery administration.

31. The nucleic acid sequence of claim 19, wherein the adenoviral protein is of an adenovirus of serotype 5, serotype 3, or serotype 35.

32.-34. (canceled)

35. A method of treating a patient who has colorectal cancer or who is considered at risk of developing colorectal cancer, the method comprising administering to the patient a nucleic acid sequence comprising a first regulatory sequence comprising SEQ ID NO:1 or a biologically active variant thereof and, operably linked thereto, a heterologous gene encoding an anti-colorectal cancer agent.

36. The method of claim 35, wherein the patient is a human and the method further comprises the step of identifying the human in need of treatment.

37. The method of claim 35, wherein the heterologous gene encodes an E1A.

38. The method of claim 35, wherein the nucleic acid sequence is contained within an expression vector.

39. The method of claim 38, wherein the expression vector is an adenoviral vector.

40. The method of claim 35, further comprising subjecting the patient to a second treatment regime.

41. The method of claim 40, wherein the second treatment regime comprises administration of an adjuvant chemotherapeutic agent selected from the group consisting of 5-fluorouracil (5-FU), capceitabine (Xeloda™), leucovorin (VL; folinic acid), and oxaliplatin (Eloxatin™).
